(19) United States
(12) Patent Application Publication
(10) Pub. No.: US 2019/0328719 A1
(43) Pub. Date: Oct. 31, 2019

(54) METHODS OF TREATING DEVELOPMENTAL DISORDERS AND/OR SEIZURE DISORDERS WITH FLUPIRTINE

(71) Applicant: Ovid Therapeutics Inc., New York, NY (US)

(72) Inventor: Matthew DURING, Weston, CT (US)

(21) Appl. No.: 16/447,010

(22) Filed: Jun. 20, 2019

Related U.S. Application Data
(63) Continuation of application No. 16/211,742, filed on Dec. 6, 2018, now abandoned.
Application No. 16/211,742 is a continuation of application No. 15/819,081, filed on Nov. 21, 2017, now abandoned.
(60) Provisional application No. 62/425,309, filed on Nov. 22, 2016.

(51) Int. Cl. A61K 31/44; A61K 9/00; A61K 45/06; A61K 31/27; A61K 31/437; A61K 31/57

(52) U.S. Cl. CPC A61K 31/44; A61K 9/0019; A61K 31/57; A61K 31/27; A61K 31/437; A61K 45/06

(57) ABSTRACT

Methods of treating developmental disorders and/or seizure disorders with flupirtine or a pharmaceutically acceptable salt thereof are provided. The methods provide therapeutic compositions that may be used to improve one or more symptoms of the developmental disorder and/or seizure disorder.

### CROSS REFERENCE TO RELATED APPLICATIONS

This application is a continuation application of U.S. patent application Ser. No. 16/211,742, filed Dec. 6, 2018, which is a continuation application of U.S. patent application Ser. No. 15/819,081, filed Nov. 21, 2017, which claims benefit of and priority to U.S. Provisional Application No. 62/425,309, filed Nov. 22, 2016, and which are incorporated herein by reference in their entireties.

### TECHNICAL FIELD

Methods of treating developmental disorders and/or seizure disorders with flupirtine are provided.

### BACKGROUND

Flupirtine is an aminopyridine known to function as a centrally acting non-opioid analgesic. It is a selective neuronal potassium channel opener that also has NMDA receptor antagonist and GABA_{A} receptor modulatory properties. Flupirtine is {2-amino-6-[(4-fluorobenzyl)amino]pyridin-3-yl}carbamate. Flupirtine is a base and poorly water soluble. It is available as a water soluble maleate salt. Flupirtine has been used in the treatment and prevention of acute and chronic pain including neuropathic pain, nerve pain, cancer pain, vasomotor and migraine headaches, post-operative pain, post-traumatic pain, burn pain, erosion pain, dysmenorrhea, dental pain and the pain associated with degenerative and inflammatory joint disease. It has muscle relaxant properties and has been used for treatment and prevention of muscular tension, muscle spasm and muscle stiffness. Flupirtine reportedly exerts cyto- and neuroprotective effects and may be used in the treatment and prevention of neurodegenerative disorders such as Parkinson's disease, dementia including Alzheimer's disease, Huntington's chorea, multiple sclerosis, amyotrophic lateral sclerosis, encephalopathy including AIDS related encephalopathy, Creutzfeldt-Jakob disease including classical and new-variant types and Batten disease. See, e.g., US Application Publication No. 2012/0142743.

Treatments for developmental disorders such as Autistic Spectrum Disorder, Dravet syndrome, Rett syndrome, Angelman syndrome, Fragile X syndrome, and Fragile X-associated tremor/ataxia syndrome are limited. Angelman syndrome is a neurodevelopmental disorder caused by loss of function of the UBE3A gene encoding a ubiquitin E3 ligase. Motor dysfunction is a characteristic feature of Angelman syndrome, but neither the mechanisms of action nor effective therapeutic strategies have yet been elucidated.

Fragile X syndrome may be the most common genetic cause of intellectual disability and the most common single-gene cause of autism. It is caused by mutations on the fragile X mental retardation gene (FMR1) and lack of fragile X mental retardation protein, which in turn, leads to decreased inhibition of translation of many synaptic proteins. The main efforts have focused on metabotropic glutamate receptor (mGluR) targeted treatments; however, investigation on the gamma-aminobutyric acid (GABA) system and its potential as a targeted treatment is less emphasized. The fragile X mouse models (Fmr1-knock out) show decreased GABA subunit receptors, decreased synthesis of GABA, increased catabolism of GABA, and overall decreased GABAergic input in many regions of the brain. These symptoms are also observed in individuals with autism and other neurodevelopmental disorders, therefore the targeted treatments for Fragile X syndrome are leading the way in the treatment of other neurodevelopmental syndromes and autism. Potential GABAergic treatments, such as riluzole, gaboxadol, tiagabine, and vigabatrin have been discussed. However, further studies are needed to determine the safety and efficacy of GABAergic treatments for Fragile X syndrome.

Fragile X-associated tremor/ataxia syndrome (FXTAS) is a late-onset disorder, usually occurring after age 50. Mutations in the FMR1 gene increase the risk of developing FXTAS. The mutation relates to a DNA segment known as a CGG triplet repeat which is expanded within the FMR1 gene. Normally, this DNA segment is repeated from 5 to about 40 times. In people with FXTAS the CGG segment may be repeated 55 to 200 times. This mutation is known as an FMR1 gene premutation. An expansion of more than 200 repeats, a full mutation, causes Fragile X syndrome discussed above. FXTAS is typically characterized by problems with movement and thinking ability (cognition). FXTAS signs and symptoms usually worsen with age. Affected individuals have areas of damage in the cerebellum, the area of the brain that controls movement. Characteristic features of FXTAS are intention tremor, which is trembling or shaking of a limb when trying to perform a voluntary movement such as reaching for an object, and problems with coordination and balance (ataxia). Many affected individuals develop other movement problems, such as parkinsonism, which includes tremors when not moving (resting tremor), rigidity, and unusually slow movement (bradykinesia). In addition, affected individuals may have reduced sensation, numbness or tingling, pain, or muscle weakness in the lower limbs, and inability to control the bladder or bowel. Other symptoms may include chronic pain syndromes, such as fibromyalgia and chronic migraine, hypothyroidism, hypertension, insomnia, sleep apnea, vertigo, olfactory dysfunction, and hearing loss. People with FXTAS commonly have cognitive disabilities such as short-term memory loss and loss of executive function, which is the ability to plan and implement actions and develop problem-solving strategies. Loss of this function impairs skills such as impulse control, self-monitoring, focusing attention appropriately, and cognitive flexibility. Many people with FXTAS experience psychiatric symptoms such as anxiety, depression, moodiness, or irritability.

There is currently no targeted therapeutic intervention that can arrest or reverse the pathogenesis of FXTAS. However a number of treatment approaches of potential symptomatic benefit have been suggested. Primidone, beta-blockers such as propanolol, topiramate, carbidopa/levodopa, and benzodiazepines have been suggested to control tremors associated with FXTAS; botulinum toxin for involuntary muscle activities, such as dystonia and spasticity; carbidopa/levodopa, amantadine and buspirone for ataxia; cholinesterase inhibitors such as donepezil, and memantine (an NMDA antagonist) for cognitive deficits and dementia; and antidepressants and antipsychotics for psychiatric symptoms. See, e.g., Hagerman, et al., Clin Interv Aging. 2008 June; 3(2): 251-262.

Rett syndrome is a neurodevelopmental disorder that typically affects girls. It is characterized by normal early growth and development followed by a slowing of development, loss of purposeful use of the hands, distinctive hand movements, slowed brain and head growth, problems with walking, seizures, and intellectual disability. Nearly all cases of Rett syndrome are caused by a mutation in the methyl CpG binding protein 2, or MECP2 gene. The MECP2 gene contains instructions for the synthesis of methyl cytosine binding protein 2 (MeCP2), which is utilized in brain development and acts as one of the many biochemical switches that can either increase or decrease gene expression. The main diagnostic criteria or symptoms include partial or complete loss of acquired purposeful hand skills, partial or complete loss of acquired spoken language, repetitive hand movements (such has hand wringing or squeezing, clapping or rubbing), and gait abnormalities, including toe-walking or an unsteady, wide-based, stiff-legged walk. Supportive criteria are not required for a diagnosis of Rett syndrome but may occur in some individuals. In addition, these symptoms, which vary in severity from child to child, may not be observed in very young children but may develop with age. A child with supportive criteria but none of the essential criteria does not have Rett syndrome. Supportive criteria include scoliosis, teeth-grinding, small cold hands and feet in relation to height, abnormal sleep patterns, abnormal muscle tone, heart abnormalities, inappropriate laughing or screaming, intense eye communication, and diminished response to pain.

There is no cure for Rett syndrome. Treatment for the disorder is symptomatic, focusing on the management of symptoms, and supportive, requiring a multidisciplinary approach. Medication may be needed for breathing irregularities and motor difficulties, and anticonvulsant drugs may be used to control seizures.

Dravet syndrome, also known as Severe Myoclonic Epilepsy of Infancy (SMEI) is a severe form of intractable epilepsy that begins in infancy with febrile seizures. Later, patients also manifest other seizure types, including absence, myoclonic, and partial seizures. The EEG is often normal at first, but later characteristically shows generalized spike-wave activity. Psychomotor development stagnates around the second year of life, and affected individuals show subsequent mental decline and other neurologic manifestations. Dravet syndrome is associated with ataxia, slowed psychomotor development, and mental decline, and is often refractory to medication. Dravet syndrome has been associated with mutation of the SCN1A gene on chromosome 2q24.

Accordingly, there remains a need for effective treatments of patients with developmental disorders, such as Autistic Spectrum Disorder, pervasive developmental disorder, Autism, Angelman syndrome, Fragile X syndrome, Fragile X-associated tremor/ataxia syndrome (FXTAS), Rett syndrome, Asperger's syndrome, Childhood Disintegrative Disorder, Attention-deficit/hyperactivity disorder (ADHD), Prader-Willi Syndrome, Landau-Kleffner Syndrome, Rasmussen's syndrome, Dravet syndrome, Tardive Dyskinesia, Williams Syndrome and/or seizure disorders per se, and/or seizure disorders associated with any of the foregoing developmental disorders. Examples of seizure disorders include epilepsy, epilepsy with generalized tonic-clonic seizures, epilepsy with myoclonic absences, frontal lobe epilepsy, temporal lobe epilepsy, Dravet syndrome, Landau-Kleffner Syndrome, Rasmussen's syndrome, Doose syndrome, CDKL5 disorder, West syndrome, Lennox-Gastaut syndrome (LGS), Rett syndrome, Ohtahara syndrome, CDKL5 disorder, childhood absence epilepsy, essential tremor, and acute repetitive seizures, benign rolandic epilepsy, status epilepticus, refractory status, epilepticus, super-refractory status epilepticus (SRSE), PCDH19 pediatric epilepsy, increased seizure activity or breakthrough seizures (increased seizure activity; also called serial or cluster seizures).

### SUMMARY

Methods of treating a developmental disorder described herein include administering flupirtine or a pharmaceutically acceptable salt thereof to a patient in need thereof to provide improvement in one or more symptoms of the disorder. In embodiments, methods of treating a developmental disorder described herein include administering flupirtine or a pharmaceutically acceptable salt thereof to a patient in need thereof to provide improvement in next day functioning of the patient. Methods of treating a seizure disorder described herein include administering flupirtine or a pharmaceutically acceptable salt thereof to a patient in need thereof to provide improvement in one or more symptoms of the disorder. In embodiments, methods of treating a seizure disorder described herein include administering flupirtine or a pharmaceutically acceptable salt thereof to a patient in need thereof to provide improvement in next day functioning of the patient. In embodiments, flupirtine or a pharmaceutically acceptable salt thereof is administered in combination with one or more of retigabine, N-[2-Amino-4-[[(2,4,6-trimethylphenyl)methyl]-amino]phenyl]carbamic acid-ethyl ester, gaboxadol, pipradrol, ganaxolone or allopregnanolone, or a pharmaceutically acceptable salt of any of the preceding.

In embodiments, the developmental disorder may be an Autistic Spectrum Disorder, pervasive developmental disorder, Autism, Angelman syndrome, Fragile X syndrome, Fragile X-associated tremor/ataxia syndrome (FXTAS), Rett syndrome, Asperger's syndrome, Childhood Disintegrative Disorder, Attention-deficit/hyperactivity disorder (ADHD), Prader-Willi Syndrome, Landau-Kleffner Syndrome, Rasmussen's syndrome, Dravet syndrome, Tardive Dyskinesia, Williams Syndrome and/or seizure disorders per se, and/or seizure disorders associated with any of the foregoing developmental disorders. Examples of seizure disorders include epilepsy, epilepsy with generalized tonic-clonic seizures, epilepsy with myoclonic absences, frontal lobe epilepsy, temporal lobe epilepsy, Landau-Kleffner Syndrome, Dravet syndrome, Rasmussen's syndrome, Doose syndrome, CDKL5 disorder, West syndrome, Lennox-Gastaut syndrome (LGS), Rett syndrome, Ohtahara syndrome, CDKL5 disorder, childhood absence epilepsy, essential tremor, and acute repetitive seizures, benign rolandic epilepsy, status epilepticus, refractory status epilepticus, super-refractory status epilepticus (SRSE), PCDH19 pediatric epilepsy, increased seizure activity or breakthrough seizures (increased seizure activity, also called serial or cluster seizures). In embodiments, the seizure disorder is status epilepticus.

In embodiments, a combination of flupirtine or a pharmaceutically acceptable salt thereof and a benzodiazepine, or another potassium channel opener, may be administered to a patient in need thereof.

### DETAILED DESCRIPTION

Described herein are methods of treating a developmental disorder that include administering flupirtine or a pharmaceutically acceptable salt thereof to a patient in need thereof to provide improvement in one or more symptoms of the disorder. In embodiments, methods of treating a developmental disorder described herein include administering flupirtine or a pharmaceutically acceptable salt thereof to a patient in need thereof to provide improvement in next day functioning of the patient. Described herein are methods of treating a seizure disorder that include administering flupirtine or a pharmaceutically acceptable salt thereof to a patient in need thereof to provide improvement in one or more symptoms of the disorder. In embodiments, methods of treating a seizure disorder described herein include administering flupirtine or a pharmaceutically acceptable salt thereof to a patient in need thereof to provide improvement in next day functioning of the patient. In embodiments, methods of treating a developmental disorder and/or a seizure disorder include administering flupirtine or a pharmaceutically acceptable salt thereof to a patient in need thereof. In embodiments, methods of treating a developmental disorder and/or a seizure disorder include administering a pharmaceutical composition containing flupirtine or a pharmaceutically acceptable salt thereof to a patient in need thereof. In embodiments, methods of treating a developmental disorder and/or a seizure disorder include administering a pharmaceutical composition containing flupirtine or a pharmaceutically acceptable salt thereof to a patient in need thereof to provide improvement in one or more symptoms of the disorder. In embodiments, methods of treating a developmental disorder and/or a seizure disorder described herein include administering a pharmaceutical composition containing flupirtine or a pharmaceutically acceptable salt thereof to a patient in need thereof to provide improvement in next day functioning of the patient.

In embodiments, the developmental disorder may be an Autistic Spectrum Disorder, pervasive developmental disorder, autism, Angelman syndrome, Fragile X syndrome, Fragile X-associated tremor/ataxia syndrome (FXTAS), Rett syndrome, Asperger's syndrome, Childhood Disintegrative Disorder, Attention-deficit/hyperactivity disorder (ADHD), Prader-Willi Syndrome, Landau-Kleffner Syndrome, Rasmussen's syndrome, Dravet syndrome, Tardive Dyskinesia, Williams Syndrome and/or seizure disorders per se, and/or seizure disorders independent of, or associated with, any of the foregoing developmental disorders.

Examples of seizure disorders include epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, increased seizure activity or breakthrough seizures (also called serial or cluster seizures). In embodiments, the seizure disorder is associated with a sodium channel protein type 1 subunit alpha (Scn1a)-related disorder.

In embodiments, the seizure disorder is status epilepticus (SE). SE is characterized by an epileptic seizure of greater than five minutes or more than one seizure within a five-minute period without the person returning to normal between them. SE can be a dangerous condition that can lead to mortality if treatment is delayed. SE can be convulsive, with a regular pattern of contraction and extension of the arms and legs, or non-convulsive, with a change in a person's level of consciousness of relatively long duration but without large scale bending and extension of the limbs due to seizure activity. Convulsive SE (CSE) may be further classified into (a) tonic-clonic SE, (b) tonic SE, (c) clonic SE and (d) myoclonic SE. Non-convulsive SE (NCSE) is characterized by abnormal mental status, unresponsiveness, ocular motor abnormalities, persistent electrographic seizures, and possible response to anticonvulsants.

In embodiments, the developmental disorder is a pervasive developmental disorder not otherwise characterized (PDD-NOS). Symptoms of PDD-NOS can vary widely from one child to the next. Overall, child with PDD-NOS can be characterized as having impaired social interaction, better language skills than children with autistic disorder but not as good as those with Asperger's syndrome, fewer repetitive behaviors than children with Asperger's syndrome or autistic disorder, and a later age of onset.

In embodiments, the developmental disorder is autism. In embodiments, the developmental disorder is Angelman syndrome. In embodiments the development disorder is Fragile X syndrome. In embodiments, the developmental disorder is Fragile X-associated tremor/ataxia syndrome (FXTAS). In embodiments, the developmental disorder is Rett syndrome.

Many pharmaceutical products are administered as a fixed dose, at regular intervals, to achieve therapeutic efficacy. Duration of action is typically reflected by a drug's plasma half-life. Since efficacy is often dependent on sufficient exposure within the central nervous system administration of CNS drugs with a short half-life may require frequent maintenance dosing. The half-life of flupirtine following intravenous administration is about 1.8 hours, while the plasma elimination half-life in healthy young volunteers following single dose administration of flupirtine by the intravenous, oral and rectal routes is about 8.5, 9.6 and 10.7 hours, respectively. A single oral dose of flupirtine 100 mg or 200 mg administered to healthy volunteers appears in the plasma within about 15-30 minutes resulting in peak plasma concentrations (Cₘₐₓ) of approximately 0.8 and 2.0 mg/L at 1.6 to 2 hours (Tₘₐₓ) post dose. Plasma flupirtine concentrations are linearly related to dose over the range of 50 to 300 mg. Rectal administration of flupirtine maleate 150 mg results in a Cₘₐₓ of about 0.89 mg/L at 5.7 hours post dose. Bioavailability in comparison with an intravenous dose of flupirtine tartrate 80 mg is about 90% to 100% for the oral dose and about 72.5% for the rectal dose. Plasma flupirtine concentrations reach steady-state after 2 days when receiving flupirtine 75 or 150 mg at 12 hour intervals.

In embodiments, flupirtine may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, sulfuric, sulfamic, phosphoric or nitric acid addition salts may be used.

In embodiments, methods of treating a developmental disorder and/or a seizure disorder are provided which include administering flupirtine or a pharmaceutically acceptable salt thereof to a patient in need thereof in an amount of about 20 mg to about 2000 mg. In embodiments, flupirtine or a pharmaceutically acceptable salt thereof is administered to a patient in an amount which is effective (an “effective amount”) to provide improvement in one or more symptoms of the disorders mentioned above. In embodiments, methods of treating a disorder described herein include administering flupirtine or a pharmaceutically acceptable salt thereof to a patient in need thereof in an amount which is effective to provide improvement in next day functioning of the patient.

In embodiments, the amount of flupirtine or a pharmaceutically acceptable salt thereof administered daily can be between about 10 mg and 1000 mg or more. For example, the daily dosage can be 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg or 1600 mg flupirtine or a pharmaceutically acceptable salt thereof. In general, the daily dosage should not exceed 1600 mg. However, there are situations when amounts greater than 1600 mg can be administered. In embodiments, an adult dose can be about 300-400 mg per day and can be increased to 600 mg per day. Dosages can be lower for infants and children than for adults. In embodiments, a pediatric dose can be about 10-500 mg per day in 3 to 4 divided doses.

In embodiments, flupirtine or a pharmaceutically acceptable salt thereof can be administered one, two, three, four or more times daily in divided doses. In embodiments, flupirtine or a pharmaceutically acceptable salt thereof is administered via a pharmaceutical composition. Pharmaceutical compositions herein encompass dosage forms. Dosage forms herein encompass unit doses. In embodiments, as discussed below, various dosage forms including conventional formulations and modified release formulations can be administered one or more times daily. Any suitable route of administration may be utilized, e.g., oral, rectal, nasal, pulmonary, vaginal, sublingual, transdermal, intravenous, intraarterial, intramuscular, intraperitoneal and subcutaneous routes. Suitable dosage forms include tablets, capsules, oral liquids, powders, aerosols, transdermal modalities such as topical liquids, patches, creams and ointments, parenteral formulations and suppositories. In embodiments, rectal suppositories containing flupirtine can be administered in a dose range of about 450-600 mg per day in adults and about 10-400 mg per day in infants and children. Flupirtine may be administered intravenously to adults, infants and children, e.g., to treat status epilepticus. In embodiments, unit doses in connection with any route of administration can include 0.005 mg, 0.01 mg, 0.025 mg, 0.05 mg, 0.075 mg, 0.1 mg, 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg or 500 mg flupirtine or a pharmaceutically acceptable salt thereof. In embodiments, such amounts may be administered one or more times daily.

In embodiments, methods of treating a developmental disorder and/or a seizure disorder are provided which include administering to a patient in need thereof a pharmaceutical composition including flupirtine or a pharmaceutically acceptable salt thereof wherein the composition provides improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof wherein the composition provides improvement in one or more symptoms of the disorder for more than 8, 10, 12, 14, 16, 18, 20, 22 or 24 hours after administration to the patient. In embodiments, the pharmaceutical compositions provide improvement of next day functioning of the patient. For example, the pharmaceutical compositions may provide improvement in one or more symptoms of the disorder for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

In embodiments, flupirtine or a pharmaceutically acceptable salt thereof is administered in combination with one or more of retigabine, N-[2-Amino-4-[[(2,4,6-trimethylphenyl)methyl]amino]phenyl]carbamic acid-ethyl ester, gaboxadol, pipradrol, ganaxolone or allopregnanolone, or a pharmaceutically acceptable salt of any of the preceding. In embodiments, a combination of flupirtine or a pharmaceutically acceptable salt thereof, and a benzodiazepine, or another potassium channel opener, may be administered to a patient in need thereof. Examples of benzodiazepines are clonazepam, lorazepam, oxazepam, diazepam, chlordiazepoxide, clorazepate, flurazepam, triazolam, temazepam, midazolam and alprazolam.

In embodiments, methods of treating a developmental disorder and/or a seizure disorder are provided which include administering flupirtine or a pharmaceutically acceptable salt thereof in an amount discussed above and retigabine or a pharmaceutically acceptable salt thereof to a patient in need thereof. Retigabine is also known as ezogabine. The chemical name of retigabine is N-[2-amino-4-(4-fluorobenzylamino)-phenyl] carbamic acid ethyl ester. Retigabine is a potassium channel opener.

Pharmaceutically acceptable acid salts of retigabine include acid addition salts formed from acids that provide non-toxic anions. Examples of pharmaceutically acceptable salts include but are not limited to, acetate, aspartate, benzoate, bicarbonate, carbonate, bisulfate, sulfate, chloride, bromide, benzene sulfonate, methyl sulfonate, phosphate, acid phosphate, lactate, maleate, malate, malonate, fumarate, lactate, tartrate, borate, camsylate, citrate, edisylate, esylate, formate, fumarate, gluceptate, glucuronate, gluconate oxalate, palmitate, pamoate, saccharate, stearate, succinate, tartrate, tosylate, and trifluoroacetate salts, and the like. The salts may also be hemi-salts, including but not limited to a hemi-sulfate salt, and the like.

Retigabine or a pharmaceutically acceptable salt thereof can be administered to a patient in an amount of about 1 mg to about 1600 mg. Retigabine or a pharmaceutically acceptable salt thereof may be co-administered simultaneously with flupirtine or a pharmaceutically acceptable salt thereof or at spaced apart intervals. In embodiments, 1 mg, 2 mg, 3 mg, 4 mg, 5 mg, 6 mg, 7 mg, 8 mg, 9 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1125 mg, 1150 mg, 1175 mg, or 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, or 1600 mg, retigabine or a pharmaceutically acceptable salt thereof can be administered to a patient per day. In embodiments, an initial dosage of retigabine or a pharmaceutically acceptable salt thereof can be 100 mg three times daily (300 mg per day). In embodiments, the dosage can be increased gradually at weekly intervals by, e.g., 50 mg 3 times daily (increase in the daily dose of 150 mg per day) up to a maintenance dosage of 200 mg to 400 mg 3 times daily (600 mg to 1,200 mg per day), based on individual patient response and tolerability. Dosages can be lower for infants and children than for adults. In embodiments, unit doses can include 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg or 500 mg retigabine or a pharmaceutically acceptable salt thereof. Flupirtine or a pharmaceutically acceptable salt thereof, and retigabine or a pharmaceutically acceptable salt thereof may be administered in separate dosage forms or combined in one dosage form.

After both single and multiple oral doses, retigabine is rapidly absorbed with median time to maximum plasma concentration (Cₘₐₓ) values generally between about 0.5 and about 2 hours. Retigabine has a median plasma half-life of about 6-8 hours. Absolute oral bioavailability of retigabine relative to an intravenous dose of retigabine is approximately 60%. Retigabine and its N-acetyl metabolite have similar elimination half-lives (t/2) of about 7 to 11 hours. The clearance of retigabine following intravenous dosing is approximately 0.4 to 0.6 L/hr/kg. The pharmacokinetics of retigabine are linear over a dose range 200-400 mg three times daily, with 35-50% between-subject variability.

In embodiments, flupirtine or a pharmaceutically acceptable salt thereof and retigabine or a pharmaceutically acceptable salt thereof are administered to a patient in an amount which is effective to provide improvement in one or more symptoms of the disorders mentioned above. In embodiments, methods of treating a developmental disorder and/or a seizure disorder are provided which include administering to a patient in need thereof flupirtine or a pharmaceutically acceptable salt thereof and retigabine or a pharmaceutically acceptable salt thereof wherein improvement in one or more symptoms of the disorder occurs for more than 6 hours after administration to the patient. In embodiments, methods of treating a developmental disorder and/or a seizure disorder are provided which include administering to a patient in need thereof flupirtine or a pharmaceutically acceptable salt thereof and retigabine or a pharmaceutically acceptable salt thereof wherein improvement in one or more symptoms of the disorder occurs for more than 8, 10, 12, 14, 16, 18, 20, 22 or 24 hours after administration to the patient. In embodiments, methods of treating a disorder described herein include administering flupirtine or a pharmaceutically acceptable salt thereof and retigabine or a pharmaceutically acceptable salt thereof to a patient in need thereof in an amount which is effective to provide improvement in next day functioning of the patient. For example, improvement in one or more symptoms of the disorder are provided for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

In embodiments, methods of treating a developmental disorder and/or a seizure disorder are provided which include administering to a patient in need thereof a pharmaceutical composition including flupirtine or a pharmaceutical salt thereof, and retigabine or a pharmaceutically acceptable salt thereof. In embodiments, methods of treating a developmental disorder and/or a seizure disorder are provided which include administering to a patient in need thereof a pharmaceutical composition including flupirtine or a pharmaceutical salt thereof, and retigabine or a pharmaceutically acceptable salt thereof wherein the composition provides improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and a pharmaceutical composition including retigabine or a pharmaceutically acceptable salt thereof. In embodiments, methods of treating a developmental disorder and/or a seizure disorder are provided which include administering to a patient in need thereof a pharmaceutical composition including flupirtine or a pharmaceutical salt thereof, and a pharmaceutical composition including retigabine or a pharmaceutically acceptable salt thereof wherein the compositions provide improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and retigabine or a pharmaceutically acceptable salt thereof wherein the composition provides improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutical salt thereof, and a pharmaceutical composition including retigabine or a pharmaceutically acceptable salt thereof wherein the compositions provide improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and retigabine or a pharmaceutically acceptable salt thereof wherein the composition provides improvement in one or more symptoms of the disorder for more than 8, 10, 12, 14, 16, 18, 20, 22 or 24 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and a pharmaceutical composition including retigabine or a pharmaceutically acceptable salt thereof wherein the compositions provide improvement in one or more symptoms of the disorder for more than 8, 10, 12, 14, 16, 18, 20, 22 or 24 hours after administration to the patient.

In embodiments, flupirtine or a pharmaceutically acceptable salt thereof, and retigabine or a pharmaceutically acceptable salt thereof are administered separately. In embodiments, flupirtine or a pharmaceutically acceptable salt thereof and retigabine or a pharmaceutically acceptable salt thereof are administered together. In embodiments, flupirtine or a pharmaceutically acceptable salt thereof and retigabine or a pharmaceutically acceptable salt thereof are administered in a single pharmaceutical composition. In embodiments, flupirtine or a pharmaceutically acceptable salt thereof and retigabine or a pharmaceutically acceptable salt thereof are administered in separate pharmaceutical compositions. In embodiments, flupirtine or a pharmaceutically acceptable salt thereof and retigabine or a pharmaceutically acceptable salt thereof may be administered at spaced apart intervals.

In embodiments, methods of treating a developmental disorder and/or a seizure disorder are provided which include administering flupirtine or a pharmaceutically acceptable salt thereof in an amount discussed above, and N-[2-Amino-4-[[(2,4,6-trimethylphenyl)methyl]amino]phenyl]-carbamic acid-ethyl ester or a pharmaceutically acceptable salt thereof to a patient. N-[2-Amino-4-[[(2,4,6-trimethylphenyl)methyl]amino]phenyl]-carbamic acid-ethyl ester may be represented by the formula:

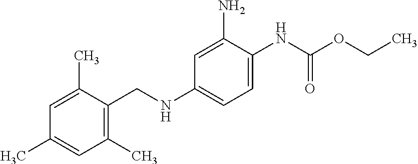 [SMILES: CCOC(=O)Nc1ccc(NCc2c(C)cc(C)cc2C)cc1N]

For example, in embodiments, methods of treating a developmental disorder and/or a seizure disorder are provided which include administering to a patient in need thereof flupirtine or a pharmaceutical salt thereof, and N-[2-Amino-4-[[(2,4,6-trimethylphenyl)methyl]amino]phenyl]-carbamic acid-ethyl ester or a pharmaceutically acceptable salt thereof. In embodiments, methods of treating a developmental disorder and/or a seizure disorder are provided which include administering to a patient in need thereof a pharmaceutical composition including flupirtine or a pharmaceutical salt thereof, and N-[2-Amino-4-[[(2,4,6-trimethylphenyl)methyl]amino]phenyl]-carbamic acid-ethyl ester or a pharmaceutically acceptable salt thereof. In embodiments, about 0.05 mg to about 75 mg of N-[2-Amino-4-[[(2,4,6-trimethylphenyl)methyl]amino]phenyl]-carbamic acid-ethyl ester or a pharmaceutically acceptable salt thereof are administered to a patient.

Pharmaceutically acceptable acid salts of N-[2-Amino-4-[[(2,4,6-trimethylphenyl)methyl]amino]phenyl]-carbamic acid-ethyl ester include acid addition salts formed from acids that provide non-toxic anions. Examples of pharmaceutically acceptable salts include but are not limited to, acetate, aspartate, benzoate, bicarbonate, carbonate, bisulfate, sulfate, chloride, bromide, benzene sulfonate, methyl sulfonate, phosphate, acid phosphate, lactate, maleate, malate, malonate, fumarate, lactate, tartrate, borate, camsylate, citrate, edisylate, esylate, formate, fumarate, gluceptate, glucuronate, gluconate oxalate, palmitate, pamoate, saccharate, stearate, succinate, tartrate, tosylate, and trifluoroacetate salts, and the like. The salts may also be hemi-salts, including but not limited to a hemi-sulfate salt, and the like.

In embodiments, methods of treating a developmental disorder and/or a seizure disorder are provided which include administering to a patient in need thereof flupirtine or a pharmaceutical salt thereof, and N-[2-Amino-4-[[(2,4,6-trimethylphenyl)methyl]amino]phenyl]-carbamic acid-ethyl ester or a pharmaceutically acceptable salt thereof, wherein the patient exhibits improvement in one or more symptoms of the disorder. In embodiments, the amount of flupirtine or a pharmaceutical salt thereof, and N-[2-Amino-4-[[(2,4,6-trimethylphenyl)methyl]amino]phenyl]-carbamic acid-ethyl ester or a pharmaceutically acceptable salt thereof administered is effective to provide improvement in one or more symptoms of the disorder. In embodiments, methods of treating a developmental disorder are provided which include administering to a patient in need thereof flupirtine or a pharmaceutical salt thereof, and N-[2-Amino-4-[[(2,4,6-trimethylphenyl)methyl]amino]phenyl]-carbamic acid-ethyl ester or a pharmaceutically acceptable salt thereof wherein the patient exhibits improvement in one or more symptoms of the disorder for more than 6 hours after administration to the patient. In embodiments the amount of flupirtine or a pharmaceutical salt thereof, and N-[2-Amino-4-[[(2,4,6-trimethylphenyl)methyl]amino]phenyl]-carbamic acid-ethyl ester or a pharmaceutically acceptable salt thereof is effective to provide improvement in one or more symptoms of the disorder for more than 6 hours after administration to the patient. In embodiments, flupirtine or a pharmaceutical salt thereof, in an amount described above, and about 0.05 mg to about 75 mg of N-[2-Amino-4-[[(2,4,6-trimethylphenyl)methyl]amino]phenyl]-carbamic acid-ethyl ester or a pharmaceutically acceptable salt thereof are administered to a patient.

In embodiments, methods of treating a developmental disorder and/or a seizure disorder are provided which include administering to a patient in need thereof flupirtine or a pharmaceutically acceptable salt thereof, and N-[2-Amino-4-[[(2,4,6-trimethylphenyl)methyl]amino]phenyl]-carbamic acid-ethyl ester or a pharmaceutically acceptable salt thereof wherein improvement in one or more symptoms of the disorder occurs for more than 8, 10, 12, 14, 16, 18, 20, 22 or 24 hours after administration to the patient. In embodiments, the amount of flupirtine or a pharmaceutically acceptable salt thereof, and N-[2-Amino-4-[[(2,4,6-trimethylphenyl)methyl]amino]phenyl]-carbamic acid-ethyl ester or a pharmaceutically acceptable salt thereof administered to a patient is effective to provide improvement in one or more symptoms of the disorder which occurs for more than 8, 10, 12, 14, 16, 18, 20, 22 or 24 hours after administration to the patient.

In embodiments, flupirtine or a pharmaceutically acceptable salt thereof, and N-[2-Amino-4-[[(2,4,6-trimethylphenyl)methyl]amino]phenyl]-carbamic acid-ethyl ester or a pharmaceutically acceptable salt thereof are administered separately. In embodiments, flupirtine or a pharmaceutically acceptable salt thereof, and N-[2-Amino-4-[[(2,4,6-trimethylphenyl)methyl]amino]phenyl]-carbamic acid-ethyl ester or a pharmaceutically acceptable salt thereof are administered separately, at spaced apart intervals. In embodiments, flupirtine or a pharmaceutically acceptable salt thereof and N-[2-Amino-4-[[(2,4,6-trimethylphenyl)methyl]amino]phenyl]-carbamic acid-ethyl ester or a pharmaceutically acceptable salt thereof are administered together.

In embodiments, methods of treating a developmental disorder and/or a seizure disorder are provided which include administering to a patient in need thereof a pharmaceutical composition including flupirtine or a pharmaceutical salt thereof, and N-[2-Amino-4-[[(2,4,6-trimethylphenyl)methyl]amino]phenyl]-carbamic acid-ethyl ester or a pharmaceutically acceptable salt thereof wherein the composition provides improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and N-[2-Amino-4-[[(2,4,6-trimethylphenyl)methyl]amino]phenyl]-carbamic acid-ethyl ester or a pharmaceutically acceptable salt thereof wherein the composition provides improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and N-[2-Amino-4-[[(2,4,6-trimethylphenyl)methyl]-amino]phenyl]-carbamic acid-ethyl ester or a pharmaceutically acceptable salt thereof wherein the composition provides improvement in one or more symptoms of the disorder for more than 8, 10, 12, 14, 16, 18, 20, 22 or 24 hours after administration to the patient.

In embodiments, methods of treating a developmental disorder and/or a seizure disorder are provided which include administering to a patient in need thereof a pharmaceutical composition including flupirtine or a pharmaceutical salt thereof, and a pharmaceutical composition including N-[2-Amino-4-[[(2,4,6-trimethylphenyl)methyl]amino]phenyl]-carbamic acid-ethyl ester or a pharmaceutically acceptable salt thereof wherein the compositions provide improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and a pharmaceutical composition including N-[2-Amino-4-[[(2,4,6-trimethylphenyl)methyl]amino]phenyl]-carbamic acid-ethyl ester or a pharmaceutically acceptable salt thereof wherein the compositions provide improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and a pharmaceutical composition including N-[2-Amino-4-[[(2,4,6-trimethylphenyl)methyl]-amino]phenyl]-carbamic acid-ethyl ester or a pharmaceutically acceptable salt thereof wherein the compositions provide improvement in one or more symptoms of the disorder for more than 8, 10, 12, 14, 16, 18, 20, 22 or 24 hours after administration to the patient. In embodiments, a composition containing flupirtine or a pharmaceutically acceptable salt thereof, and a composition containing N-[2-Amino-4-[[(2,4,6-trimethylphenyl)methyl]amino]phenyl]-carbamic acid-ethyl ester or a pharmaceutically acceptable salt thereof are administered separately. In embodiments, a composition containing flupirtine or a pharmaceutically acceptable salt thereof and a composition containing N-[2-Amino-4-[[(2,4,6-trimethylphenyl)methyl]amino]phenyl]-carbamic acid-ethyl ester or a pharmaceutically acceptable salt thereof are administered together. In embodiments, flupirtine or a pharmaceutically acceptable salt thereof and N-[2-Amino-4-[[(2,4,6-trimethylphenyl)methyl]amino]-phenyl]-carbamic acid-ethyl ester or a pharmaceutically acceptable salt thereof, may be administered at spaced apart intervals.

In embodiments, methods of treating a developmental disorder and/or a seizure disorder include administering to a patient in need thereof flupirtine or a pharmaceutically acceptable salt thereof, and about 0.05 mg to about 75 mg of N-[2-Amino-4-[[(2,4,6-trimethylphenyl)methyl]amino]phenyl]-carbamic acid-ethyl ester or a pharmaceutically acceptable salt thereof. In embodiments, about 0.05 mg to about 75 mg of N-[2-Amino-4-[[(2,4,6-trimethylphenyl)-methyl]amino]phenyl]-carbamic acid-ethyl ester or a pharmaceutically acceptable salt thereof is administered in 24 hours. In embodiments, 0.05 mg to about 75 mg of N-[2-Amino-4-[[(2,4,6-trimethylphenyl)methyl]amino]phenyl]-carbamic acid-ethyl ester or a pharmaceutically acceptable salt thereof is administered in divided doses over 24 hours.

In embodiments, N-[2-Amino-4-[[(2,4,6-trimethylphenyl)methyl]-amino]phenyl]-carbamic acid-ethyl ester or a pharmaceutically acceptable salt thereof is administered at dosages ranging from about 0.001 mg/kg to about 10 mg/kg of body weight of a patient in need thereof, e.g., from about 0.01 mg/kg to 2.0 mg/kg at least once a day. For example, dosages may include amounts of N-[2-Amino-4-[[(2,4,6-trimethylphenyl)methyl]amino]phenyl]-carbamic acid-ethyl ester or a pharmaceutically acceptable salt thereof in the range of about, e.g., 1 mg to 30 mg, 1 mg to 20 mg, 1 mg to 15 mg, 0.01 mg to 10 mg, 0.1 mg to 15 mg, 0.15 mg to 12.5 mg, or 0.1 mg to 10 mg, or 0.2 mg to 10 mg, with doses of 0.1 mg, 0.2 mg, 0.3 mg, 0.4 mg, 0.5 mg, 0.6 mg, 0.7 mg, 0.8 mg, 0.9 mg, 1.0 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 10.5 mg, 11 mg, 11.5 mg, 12 mg, 12.5 mg, 13 mg, 13.5 mg, 14 mg, 14.5 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg or 30 mg being non-limiting examples of doses.

Typically, dosages of N-[2-Amino-4-[[(2,4,6-trimethylphenyl)methyl]-amino]phenyl]-carbamic acid-ethyl ester or a pharmaceutically acceptable salt thereof, and flupirtine or a pharmaceutically acceptable salt thereof, are administered once daily, twice daily, three times daily, four times daily or more to a patient in need thereof. The methods and compositions described herein may provide reduced dosing frequency and reduced adverse events and/or increased efficacy. In embodiments, the dosage of N-[2-Amino-4-[[(2,4,6-trimethylphenyl)methyl]amino]phenyl]-carbamic acid-ethyl ester or a pharmaceutically acceptable salt thereof can be about, e.g., 0.1-20 mg/day, or 0.2-15 mg/day, or 0.5-10 mg/day, or 1-20 mg/day, or 5-15 mg/day, or 5-20 mg/day, or 5-30 mg/day, 5-40 mg/day, 5-50 mg/day, or 5-60 mg/day or 5-70 mg/day, or 5-80 mg/day, or 5-90 mg/day or 5-100 mg/day, or 10-15 mg/day, or 10-20 mg/day, or 10-30 mg/day, 10-40 mg/day, 10-50 mg/day, or 10-60 mg/day or 10-70 mg/day, or 10-80 mg/day, 10-90 mg/day, or 10-100 mg/day, or 0.75-5 mg/day, 0.2 mg/day, 0.5 mg/day, 0.75 mg/day, 1 mg/day, 1.5 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 21 mg/day, 22 mg/day, 23 mg/day, 24 mg/day, 25 mg/day, 26 mg/day, 27 mg/day, 28 mg/day, 29 mg/day, 30 mg/day, 31 mg/day, 32 mg/day, 33 mg/day, 34 mg/day, 35 mg/day, 36 mg/day, 37 mg/day, 38 mg/day, 39 mg/day, 40 mg/day, 41 mg/day, 42 mg/day, 43 mg/day, 44 mg/day, 45 mg/day, 46 mg/day, 47 mg/day, 48 mg/day, 49 mg/day, 50 mg/day, 51 mg/day, 52 mg/day, 53 mg/day, 54 mg/day, 55 mg/day, 56 mg/day, 57 mg/day, 58 mg/day, 59 mg/day, 60 mg/day, 61 mg/day, 62 mg/day, 63 mg/day, 64 mg/day, 65 mg/day, 66 mg/day, 67 mg/day, 68 mg/day, 69 mg/day, 70 mg/day, 71 mg/day, 72 mg/day, 73 mg/day, 74 mg/day, 75 mg/day or multiples thereof. In embodiments, N-[2-Amino-4-[[(2,4,6-trimethylphenyl)methyl]amino]phenyl]-carbamic acid-ethyl ester or a pharmaceutically acceptable salt thereof can be administered at doses of 0.2 mg to 10 mg in infants, 0.5 mg to 20 mg in children, or 1-30 mg in adults, once, twice or three times daily.

In embodiments, the pharmaceutical compositions include 1 mg to 30 mg, 1 mg to 25 mg, 1 mg to 20 mg, 1 mg to 15 mg, 0.01 mg to 10 mg, 0.1 mg to 15 mg, 0.15 mg to 12.5 mg, or 0.1 mg to 10 mg, or 0.2 mg to 10 mg, with doses of 0.05 mg, 0.1 mg, 0.2 mg, 0.3 mg, 0.4 mg, 0.5 mg, 0.6 mg, 0.7 mg, 0.8 mg, 0.9 mg, 1.0 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 10.5 mg, 11 mg, 11.5 mg, 12 mg, 12.5 mg, 13 mg, 13.5 mg, 14 mg, 14.5 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg or 30 mg of N-[2-Amino-4-[[(2,4,6-trimethylphenyl)methyl]amino]phenyl]-carbamic acid-ethyl ester or a pharmaceutically acceptable salt thereof.

In embodiments, the pharmaceutical compositions include 0.05 mg, 0.1 mg, 0.2 mg, 0.3 mg, 0.4 mg, 0.5 mg, 0.6 mg, 0.7 mg, 0.8 mg, 0.9 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 10.5 mg, 11 mg, 11.5 mg, 12 mg, 12.5 mg, 13 mg, 13.5 mg, 14 mg, 14.5 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg or 30 mg of N-[2-Amino-4-[[(2,4,6-trimethylphenyl)methyl]-amino]phenyl]-carbamic acid-ethyl ester or a pharmaceutically acceptable salt thereof.

In embodiments, the total amount of N-[2-Amino-4-[[(2,4,6-trimethylphenyl)methyl]amino]phenyl]-carbamic acid-ethyl ester or a pharmaceutically acceptable salt thereof administered to a subject in a 24-hour period can be 1 mg to 100 mg. In embodiments, the total amount of N-[2-Amino-4-[[(2,4,6-trimethylphenyl)methyl]amino]phenyl]-carbamic acid-ethyl ester or a pharmaceutically acceptable salt thereof administered to a subject in a 24-hour period can be 5 mg to 75 mg. In embodiments, the total amount of N-[2-Amino-4-[[(2,4,6-trimethylphenyl)methyl]amino]phenyl]-carbamic acid-ethyl ester or a pharmaceutically acceptable salt thereof administered to a subject in a 24-hour period can be 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg. In embodiments, the total amount of N-[2-Amino-4-[[(2,4,6-trimethylphenyl)methyl]amino]phenyl]-carbamic acid-ethyl ester or a pharmaceutically acceptable salt thereof or a pharmaceutically acceptable salt thereof administered to a subject in a 24-hour period can be 150 mg.

Also provided herein are methods and compositions for treating developmental disorders and/or seizure disorders by co-administering to a patient in need thereof flupirtine or a pharmaceutically acceptable salt thereof and a GABA_{A} agonist such as gaboxadol, a derivative thereof, or a pharmaceutically acceptable salt thereof. Examples of other GABA_{A} agonists include isoguvacine, muscimol, muscimol hydrobromide, piperidine-4-sulphonic acid and isonipecotic acid.

Gaboxadol (4,5,6,7-tetrahydroisoxazolo [5,4-c]pyridine-3-ol) (THIP)) is described in EP Patent No. 0000338 and in EP Patent No. 0840601, U.S. Pat. Nos. 4,278,676, 4,362,731, 4,353,910, and WO 2005/094820. Gaboxadol is a selective GABA_{A} receptor agonist with a preference for δ-subunit containing GABA_{A} receptors. In the early 1980s gaboxadol was the subject of a series of pilot studies that tested its efficacy as an analgesic and anxiolytic, as well as a treatment for tardive dyskinesia, Huntington's disease, Alzheimer's disease, and spasticity. In the 1990s gaboxadol moved into late stage development for the treatment of insomnia. The development was discontinued after the compound failed to show significant effects in sleep onset and sleep maintenance in a three-month efficacy study. Additionally, patients with a history of drug abuse who received gaboxadol experienced a steep increase in psychiatric adverse events.

In embodiments, methods of treating a developmental disorder and/or a seizure disorder are provided which include administering flupirtine or a pharmaceutically acceptable salt thereof and gaboxadol or a pharmaceutically acceptable salt thereof to a patient in need thereof. In embodiments, methods of treating a developmental disorder and/or seizure disorder include administering flupirtine or a pharmaceutically acceptable salt thereof and gaboxadol or a pharmaceutically acceptable salt thereof to a patient in need thereof to provide improvement in one or more symptoms of the disorder. In embodiments, the amount of flupirtine or a pharmaceutical salt thereof, and gaboxadol or a pharmaceutically acceptable salt thereof administered is effective to provide improvement in one or more symptoms of the disorder. In embodiments, methods of treating a developmental disorder and/or seizure disorder described herein include administering flupirtine or a pharmaceutically acceptable salt thereof and gaboxadol or a pharmaceutically acceptable salt thereof to a patient in need thereof to provide improvement in next day functioning of the patient. In embodiments, methods of treating a developmental disorder and/or seizure disorder described herein include administering flupirtine or a pharmaceutically acceptable salt thereof and gaboxadol or a pharmaceutically acceptable salt thereof to a patient in need thereof in an amount that is effective to provide improvement in next day functioning of the patient.

In embodiments the amount of flupirtine or a pharmaceutical salt thereof, and gaboxadol or a pharmaceutically acceptable salt thereof is effective to provide improvement in one or more symptoms of the disorder for more than 6 hours after administration to the patient. In embodiments, methods of treating a developmental disorder and/or a seizure disorder are provided which include administering to a patient in need thereof flupirtine or a pharmaceutically acceptable salt thereof and gaboxadol or a pharmaceutically acceptable salt thereof wherein improvement in one or more symptoms of the disorder occurs for more than 8, 10, 12, 14, 16, 18, 20, 22 or 24 hours after administration to the patient.

In embodiments, the methods and compositions described herein include pharmaceutical compositions including flupirtine or a pharmaceutically acceptable salt thereof, and/or gaboxadol or a pharmaceutically acceptable salt thereof. In embodiments, flupirtine or a pharmaceutically acceptable salt thereof, and gaboxadol or a pharmaceutically acceptable salt thereof may be administered in separate dosage forms or combined in one dosage form. In embodiments, flupirtine or a pharmaceutically acceptable salt thereof may be co-administered simultaneously with gaboxadol or a pharmaceutically acceptable salt thereof, or at spaced apart intervals.

In embodiments, methods of treating a developmental disorder and/or a seizure disorder are provided which include administering to a patient in need thereof a pharmaceutical composition including flupirtine or a pharmaceutical salt thereof, and gaboxadol or a pharmaceutically acceptable salt thereof wherein the composition provides improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and gaboxadol or a pharmaceutically acceptable salt thereof wherein the composition provides improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and gaboxadol or a pharmaceutically acceptable salt thereof wherein the composition provides improvement in one or more symptoms of the disorder for more than 8, 10, 12, 14, 16, 18, 20, 22 or 24 hours after administration to the patient.

In embodiments, methods of treating a developmental disorder and/or a seizure disorder are provided which include administering to a patient in need thereof a pharmaceutical composition including flupirtine or a pharmaceutical salt thereof, and a pharmaceutical composition including gaboxadol or a pharmaceutically acceptable salt thereof wherein the compositions provide improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and a pharmaceutical composition including gaboxadol or a pharmaceutically acceptable salt thereof wherein the compositions provide improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and a pharmaceutical composition including gaboxadol or a pharmaceutically acceptable salt thereof wherein the compositions provide improvement in one or more symptoms of the disorder for more than 8, 10, 12, 14, 16, 18, 20, 22 or 24 hours after administration to the patient. In embodiments, a composition containing flupirtine or a pharmaceutically acceptable salt thereof, and a composition containing gaboxadol or a pharmaceutically acceptable salt thereof are administered separately. In embodiments, a composition containing flupirtine or a pharmaceutically acceptable salt thereof and a composition containing gaboxadol or a pharmaceutically acceptable salt thereof are administered together. In embodiments, flupirtine or a pharmaceutically acceptable salt thereof and gaboxadol or a pharmaceutically acceptable salt thereof, may be administered at spaced apart intervals.

Gaboxadol or pharmaceutically acceptable salt thereof may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, sulfuric, sulfamic, phosphoric or nitric acid addition salts may be used.

In embodiments, gaboxadol is provided as gaboxadol monohydrate. One skilled in the art will readily understand that the amounts of active ingredient in a pharmaceutical composition will depend on the form of gaboxadol provided. For example, pharmaceutical compositions of including 5.0, 10.0, or 15.0 mg gaboxadol correspond to 5.6, 11.3, or 16.9 mg gaboxadol monohydrate.

In embodiments, gaboxadol is crystalline, such as the crystalline hydrochloric acid salt, the crystalline hydrobromic acid salt, or the crystalline zwitter ion monohydrate. In embodiments, gaboxadol is provided as a crystalline monohydrate.

Deuteration of pharmaceuticals to improve pharmacokinetics (PK), pharmacodynamics (PD), and toxicity profiles, has been demonstrated previously with some classes of drugs. Accordingly the use of deuterium enriched gaboxadol is contemplated and within the scope of the methods and compositions described herein. Deuterium can be incorporated in any position in replace of hydrogen synthetically, according to the synthetic procedures known in the art. For example, deuterium may be incorporated to various positions having an exchangeable proton, such as the amine N—H, via proton-deuterium equilibrium exchange. Thus, deuterium may be incorporated selectively or non-selectively through methods known in the art to provide deuterium enriched gaboxadol. See Journal of Labeled Compounds and Radiopharmaceuticals 19(5) 689-702 (1982).

In embodiments, methods of treating a developmental disorder and/or seizure disorder include administering to a patient in need thereof any of the previously recited amounts of flupirtine or a pharmaceutically acceptable salt thereof and about 0.05 mg to about 50 mg gaboxadol or a pharmaceutically acceptable salt thereof. In embodiments, methods of treating a developmental disorder and/or seizure disorder include administering to a patient in need thereof any of the previously recited amounts of flupirtine or a pharmaceutically acceptable salt thereof and about 0.1 mg to about 30 mg gaboxadol or a pharmaceutically acceptable salt thereof.

In embodiments, methods of treating a developmental disorder and/or seizure disorder include administering to a patient in need thereof a pharmaceutical composition including any of the previously recited amounts of flupirtine or a pharmaceutically acceptable salt thereof and about 0.05 mg to about 50 mg gaboxadol or a pharmaceutically acceptable salt thereof. In embodiments, methods of treating a developmental disorder and/or seizure disorder include administering to a patient in need thereof a pharmaceutical composition including any of the previously recited amounts of flupirtine or a pharmaceutically acceptable salt thereof and about 0.1 mg to about 30 mg gaboxadol or a pharmaceutically acceptable salt thereof.

In embodiments, the pharmaceutical compositions include any of the previously recited amounts of flupirtine or a pharmaceutically acceptable salt thereof and/or 0.1 mg to 25 mg, 0.1 mg to 20 mg, 0.1 mg to 15 mg, 0.5 mg to 25 mg, 0.5 mg to 20 mg, 0.5 to 15 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 25 mg, 2.5 mg to 20 mg, 2.5 mg to 15 mg, 3 mg to 25 mg, 3 mg to 20 mg, or 3 mg to 15 mg of gaboxadol or a pharmaceutically acceptable salt thereof.

In embodiments, the pharmaceutical compositions include any of the previously recited amounts of flupirtine or a pharmaceutically acceptable salt thereof and/or 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof.

In embodiments, the pharmaceutical compositions include any of the previously recited amounts of flupirtine or a pharmaceutically acceptable salt thereof and/or 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, 20 mg, 25 mg, or 30 mg gaboxadol or a pharmaceutically acceptable salt thereof or amounts that are multiples of such doses. In embodiments, the pharmaceutical compositions include any of the previously recited amounts of flupirtine or a pharmaceutically acceptable salt thereof and/or 2.5 mg, 5 mg, 7.5 mg, 10 mg, 15 mg, or 20 mg gaboxadol or a pharmaceutically acceptable salt thereof.

In embodiments, the total amount of flupirtine or a pharmaceutically acceptable salt thereof and gaboxadol administered to a subject in a 24-hour period is 1 mg to 2000 mg. In embodiments, the total amount of flupirtine or a pharmaceutically acceptable salt thereof and gaboxadol administered to a subject in a 24-hour period is 1 mg to 1500 mg. In embodiments, the total amount of flupirtine or a pharmaceutically acceptable salt thereof and gaboxadol administered to a subject in a 24-hour period is 1 mg to 1000 mg. In embodiments, the total amount of flupirtine or a pharmaceutically acceptable salt thereof and gaboxadol administered to a subject in a 24-hour period is 1 mg to 500 mg. In embodiments, the total amount of flupirtine or a pharmaceutically acceptable salt thereof and gaboxadol administered to a subject in a 24-hour period is 1 mg to 100 mg. In embodiments, the total amount of flupirtine or a pharmaceutically acceptable salt thereof and gaboxadol administered to a subject in a 24-hour period is 1 mg to 75 mg. In embodiments, the total amount of flupirtine or a pharmaceutically acceptable salt thereof and gaboxadol administered to a subject in a 24-hour period is 5 mg, 10 mg, 15 mg, 20 mg, 25 mg, 30 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg or 1600 mg. In embodiments, the total amount flupirtine or a pharmaceutically acceptable salt thereof and gaboxadol or a pharmaceutically acceptable salt thereof administered to a subject in a 24-hour period is 1 mg to 50 mg. In embodiments, the subject may be started at a low dose and the dosage is escalated. In this manner, it can be determined if the drugs are well tolerated in the subject. Dosages can be lower for children than for adults. In embodiments, a dose of gaboxadol for children can be 0.1 mg/kg to 1 mg/kg.

In embodiments, methods of treating a developmental disorder and/or a seizure disorder are provided which include administering flupirtine or a pharmaceutically acceptable salt thereof in an amount discussed above and pipradrol or a pharmaceutically acceptable salt thereof to a patient in need thereof.

Pipradrol is a mild central nervous system stimulant that acts on both dopamine and norepinephrine reuptake. Pipradrol was considered an “energetic” when it first came to market in the mid to late 1950's and was used for obesity, narcolepsy, and depression. Pipradrol had also been used in obstetric and gynecological practice, for example, improving nausea and vomiting, premenstrual symptoms, post-partum psychosis, and menopausal-associated depression. Although some anticonvulsant activity has been suggested, high doses of pipradrol may cause uncoordinated activity and ataxia, followed by tremors and clonic convulsions. Following the Kefauver-Harris amendments to the FDA act in 1962, pipradrol was removed from the FDA register of approved drugs.

In embodiments, methods of treating a developmental disorder and/or a seizure disorder are provided which include administering to a patient in need thereof any of the previously recited amounts of flupirtine or a pharmaceutical salt thereof, and pipradrol or a pharmaceutically acceptable salt thereof. In embodiments, methods of treating a developmental disorder and/or a seizure disorder are provided which include administering to a patient in need thereof a pharmaceutical composition including any of the previously recited amounts of flupirtine or a pharmaceutical salt thereof, and pipradrol or a pharmaceutically acceptable salt thereof. In embodiments, about 0.5 mg to about 30 mg of pipradrol or a pharmaceutically acceptable salt thereof are administered to a patient.

In embodiments, methods of treating a developmental disorder and/or a seizure disorder are provided which include administering to a patient in need thereof flupirtine or a pharmaceutical salt thereof, and pipradrol or a pharmaceutically acceptable salt thereof wherein the patient exhibits improvement in one or more symptoms of the disorder. In embodiments, methods of treating a developmental disorder and/or a seizure disorder are provided which include administering to a patient in need thereof flupirtine or a pharmaceutical salt thereof, and pipradrol or a pharmaceutically acceptable salt thereof in an amount effective to provide the patient with improvement in one or more symptoms of the disorder. In embodiments, methods of treating a developmental disorder and/or a seizure disorder are provided which include administering to a patient in need thereof flupirtine or a pharmaceutical salt thereof, and pipradrol or a pharmaceutically acceptable salt thereof wherein the patient exhibits improvement in next day functioning of the patient. In embodiments, methods of treating a developmental disorder and/or a seizure disorder are provided which include administering to a patient in need thereof flupirtine or a pharmaceutical salt thereof, and pipradrol or a pharmaceutically acceptable salt thereof an amount effective to provide improvement in next day functioning of the patient. In embodiments, methods of treating a developmental disorder and/or seizure disorder are provided which include administering to a patient in need thereof flupirtine or a pharmaceutical salt thereof, and pipradrol or a pharmaceutically acceptable salt thereof wherein the patient exhibits improvement in one or more symptoms of the disorder for more than 6 hours after administration to the patient. In embodiments, methods of treating a developmental disorder and/or a seizure disorder are provided which include administering to a patient in need thereof flupirtine or a pharmaceutically acceptable salt thereof, and pipradrol or a pharmaceutically acceptable salt thereof wherein improvement in one or more symptoms of the disorder occurs for more than 8, 10, 12, 14, 16, 18, 20, 22 or 24 hours after administration to the patient. In embodiments, any of the previously recited amounts of flupirtine or a pharmaceutical salt thereof, and about 0.5 mg to about 30 mg of pipradrol or a pharmaceutically acceptable salt thereof are administered to a patient.

In embodiments, methods of treating a developmental disorder and/or a seizure disorder are provided which include administering to a patient in need thereof a pharmaceutical composition including flupirtine or a pharmaceutical salt thereof, and pipradrol or a pharmaceutically acceptable salt thereof. In embodiments, methods of treating a developmental disorder and/or a seizure disorder are provided which include administering to a patient in need thereof a pharmaceutical composition including flupirtine or a pharmaceutical salt thereof, and pipradrol or a pharmaceutically acceptable salt thereof wherein the composition provides improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and a pharmaceutical composition including pipradrol or a pharmaceutically acceptable salt thereof. In embodiments, methods of treating a developmental disorder and/or a seizure disorder are provided which include administering to a patient in need thereof a pharmaceutical composition including flupirtine or a pharmaceutical salt thereof, and a pharmaceutical composition including pipradrol or a pharmaceutically acceptable salt thereof wherein the compositions provide improvement in one or more symptoms of the disorder.

In embodiments, methods of treating a developmental disorder and/or a seizure disorder are provided which include administering to a patient in need thereof a pharmaceutical composition including flupirtine or a pharmaceutical salt thereof, and pipradrol or a pharmaceutically acceptable salt thereof wherein the composition provides improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutical salt thereof, and a pharmaceutical composition including pipradrol or a pharmaceutically acceptable salt thereof wherein the compositions provide improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and pipradrol or a pharmaceutically acceptable salt thereof wherein the composition provides improvement in one or more symptoms of the disorder for more than 8, 10, 12, 14, 16, 18, 20, 22 or 24 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and a pharmaceutical composition including pipradrol or a pharmaceutically acceptable salt thereof wherein the compositions provide improvement in one or more symptoms of the disorder for more than 8, 10, 12, 14, 16, 18, 20, 22 or 24 hours after administration to the patient.

In embodiments, methods of treating a disorder described herein include administering flupirtine or a pharmaceutically acceptable salt thereof and pipradrol to a patient in need thereof in an amount which is effective to provide improvement in next day functioning of the patient. In embodiments, methods of treating a developmental disorder and/or a seizure disorder are provided which include administering to a patient in need thereof a pharmaceutical composition including flupirtine or a pharmaceutical salt thereof, and pipradrol or a pharmaceutically acceptable salt thereof wherein the composition provides improvement in next day functioning of the patient. In embodiments, methods of treating a developmental disorder and/or a seizure disorder are provided which include administering to a patient in need thereof a pharmaceutical composition including flupirtine or a pharmaceutical salt thereof, and a pharmaceutical composition including pipradrol or a pharmaceutically acceptable salt thereof wherein the compositions provide improvement in next day functioning of the patient.

In embodiments, flupirtine or a pharmaceutically acceptable salt thereof, and pipradrol or a pharmaceutically acceptable salt thereof may be administered in separate dosage forms or combined in one dosage form. In embodiments, flupirtine or a pharmaceutically acceptable salt thereof may be co-administered simultaneously with pipradrol or a pharmaceutically acceptable salt thereof, or at spaced apart intervals.

In embodiments, methods of treating a developmental disorder and/or a seizure disorder include administering to a patient in need thereof any of the previously recited amounts of flupirtine or a pharmaceutically acceptable salt thereof, and about 0.5 mg to about 30 mg of pipradrol or a pharmaceutically acceptable salt thereof. In embodiments, any of the previously recited amounts of flupirtine or a pharmaceutically acceptable salt thereof and about 0.5 mg to about 30 mg of pipradrol or a pharmaceutically acceptable salt thereof is administered in 24 hours. In embodiments, any of the previously recited amounts of flupirtine or a pharmaceutically acceptable salt thereof and about 0.5 mg to about 30 mg of pipradrol or a pharmaceutically acceptable salt thereof is administered in divided doses over 24 hours.

In embodiments, pipradrol or a pharmaceutically acceptable salt thereof is administered at dosages ranging from about 0.001 mg/kg and about 10 mg/kg of body weight of a patient in need thereof, e.g., from about 0.01 mg/kg to 2.0 mg/kg at least once a day. In embodiments, pipradrol or a pharmaceutically acceptable salt thereof can be administered in amounts between about 0.5 mg to about 30 mg per day. In embodiments, pipradrol or a pharmaceutically acceptable salt thereof can be administered in amounts between about 0.75 mg, 1.0 mg, 1.25 mg, 1.50 mg, 1.75 mg, 2 mg. 2.25 mg, 2.5 mg, 2.75 mg, 3 mg. 3.25 mg, 3.5 mg, 3.75 mg, 4 mg. 4.25 mg, 4.5 mg, 4.75 mg, 5 mg. 5.25 mg, 5.5 mg, 5.75 mg, 6 mg. 6.25 mg, 6.5 mg, 6.75 mg, 7 mg. 7.25 mg, 7.5 mg, 7.75 mg, 8 mg, 8.25 mg, 8.50 mg, 8.75 mg, 9 mg. 9.25 mg, 9.5 mg, 9.75 mg, 10 mg. 10.25 mg, 10.5 mg, 10.75 mg, 11 mg. 11.25 mg, 11.5 mg, 11.75 mg, 12 mg. 12.25 mg, 12.5 mg, 12.75 mg, 13 mg. 13.25 mg, 13.5 mg, 13.75 mg, 14 mg. 14.25 mg, 14.5 mg, 14.75 mg, 15 mg. 15.25 mg, 15.5 mg, 15.75 mg, 16 mg. 16.25 mg, 16.5 mg, 16.75 mg, 17 mg. 17.25 mg, 17.5 mg, 17.75 mg, 18 mg. 18.25 mg, 18.5 mg, 18.75 mg, 19 mg. 19.25 mg, 19.5 mg, 19.75 mg and about 20 mg per day. In embodiments, dosages may include amounts of pipradrol or a pharmaceutically acceptable salt thereof in the range of about, e.g., 1 mg to 30 mg, 1 mg to 20 mg, 1 mg to 15 mg, 0.01 mg to 10 mg, 0.1 mg to 15 mg, 0.15 mg to 12.5 mg, or 0.2 mg to 10 mg, with doses of 0.1 mg, 0.2 mg, 0.3 mg, 0.4 mg, 0.5 mg, 0.6 mg, 0.7 mg, 0.8 mg, 0.9 mg, 1.0 mg, 1.5 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 13 mg, 14 mg, 15 mg, 20 mg, 25 mg, and 30 mg being specific examples of doses. In embodiments, the dosage can be about, e.g., 0.1-20 mg/day, or 0.2-15 mg/day, or 0.5-10 mg/day, or 0.75-5 mg/day, for example 0.2 mg/day, 0.5 mg/day, 0.75 mg/day, 1 mg/day, 1.5 mg/day, 2 mg/day, 3 mg/day, 4 mg/day, 5 mg/day, 6 mg/day, 7 mg/day, 8 mg/day, 9 mg/day, or 10 mg/day. In embodiments, the subject may be started at a low dose and the dosage is escalated. In this manner, it can be determined if the drugs are well tolerated in the subject. Dosages can be lower for infants and children than for adults. In embodiments, pipradrol, or salt or a derivative or analogue thereof can be administered at doses of 0.01 mg to 1 mg in infants, 0.1 mg to 15 mg in children or 1-20 mg in adults, once daily.

In embodiments, pipradrol or a pharmaceutically acceptable salt thereof may include a racemic mixture, as well as compositions including each enantiomer individually. Compositions and methods contemplated herein may provide reduced dosing frequency, reduced adverse events and/or increased efficacy compared to those containing a racemic mixture of pipradrol. In embodiments, compositions and methods that include each enantiomer individually may provide reduced dosing frequency, reduced adverse events and/or increased efficacy compared to the minor enantiomer. Thus, for example, contemplated herein are compositions and methods of treatment that provide the S enantiomer of pipradrol or a pharmaceutically acceptable salt thereof that is substantially free of the R enantiomer. In embodiments, methods and compositions herein include the R enantiomer of pipradrol or a pharmaceutically acceptable salt thereof substantially free of the S enantiomer. By “substantially free” it is meant that the composition includes less than 50% of the minor enantiomer. In embodiments, compositions and methods herein may include less than about, e.g., 25%, 15%, 10%, 8%, 5%, 3%, 2%, or less than 1% of the minor enantiomer.

In embodiments, methods of treating a developmental disorder and/or a seizure disorder are provided which include administering flupirtine or a pharmaceutically acceptable salt thereof in an amount discussed above and ganaxolone to a patient in need thereof.

Ganaxolone is a neurosteroid and CNS-selective GABA_{A} modulator. It binds to allosteric sites of the GABA_{A} receptor to modulate and open the chloride ion channel, resulting in a hyperpolarization of the neuron. Ganaxolone is being investigated for potential medical use in the treatment of epilepsy. Trials in adults with focal onset seizures and in children with infantile spasms have reportedly been completed. Additional studies are reportedly being undertaken in patients with PCDH19 pediatric epilepsy, and behaviors in Fragile X syndrome.

In embodiments, methods of treating a developmental disorder and/or a seizure disorder are provided which include administering to a patient in need thereof any of the previously recited amounts of flupirtine or a pharmaceutical salt thereof, and ganaxolone. In embodiments, methods of treating a developmental disorder and/or a seizure disorder are provided which include administering to a patient in need thereof a pharmaceutical composition including any of the previously recited amounts of flupirtine or a pharmaceutical salt thereof, and ganaxolone. In embodiments, methods of treating a developmental disorder and/or a seizure disorder are provided which include administering to a patient in need thereof a pharmaceutical composition including any of the previously recited amounts of flupirtine or a pharmaceutical salt thereof, and a pharmaceutical composition including ganaxolone. In embodiments, about 0.5 mg to about 2000 mg of ganaxolone are administered to a patient.

In embodiments, methods of treating a developmental disorder and/or a seizure disorder are provided which include administering to a patient in need thereof flupirtine or a pharmaceutical salt thereof, and ganaxolone wherein the patient exhibits improvement in one or more symptoms of the disorder. In embodiments, methods of treating a developmental disorder and/or a seizure disorder are provided which include administering to a patient in need thereof flupirtine or a pharmaceutical salt thereof, and ganaxolone in an amount effective to provide the patient with improvement in one or more symptoms of the disorder. In embodiments, methods of treating a developmental disorder and/or a seizure disorder are provided which include administering to a patient in need thereof flupirtine or a pharmaceutical salt thereof, and ganaxolone wherein the patient exhibits improvement in one or more symptoms of the disorder for more than 6 hours after administration to the patient. In embodiments, methods of treating a developmental disorder and/or a seizure disorder are provided which include administering to a patient in need thereof flupirtine or a pharmaceutical salt thereof, and ganaxolone an amount effective to provide the patient with improvement in one or more symptoms of the disorder for more than 6 hours after administration to the patient. In embodiments, methods of treating a developmental disorder and/or a seizure disorder are provided which include administering to a patient in need thereof flupirtine or a pharmaceutically acceptable salt thereof, and ganaxolone wherein improvement in one or more symptoms of the disorder occurs for more than 8, 10, 12, 14, 16, 18, 20, 22 or 24 hours after administration to the patient. In embodiments, any of the previously recited amounts of flupirtine or a pharmaceutical salt thereof, and about 0.5 mg to about 2000 mg of ganaxolone are administered to a patient.

After both single and multiple oral doses, ganaxolone is rapidly absorbed with median time to maximum plasma concentration (Cₘₐₓ) values generally between about 1 to about 3 hours. Ganaxolone has half-life which is reportedly about 20 hours. The terminal half-life is also reportedly about 40 to 65 hours after both single and multiple dosing over a dosing range of 50-500 mg/day. Ganaxolone exhibits a linear and proportional increase in the area under the curve (AUC) and Cₘₐₓ values with increasing dose. Over the course of 14 days of administration, Cₘₐₓ values (without correction for Cₘᵢₙ) increase proportionally with dose from 32 ng/ml (50 mg group) to 106 and 376 ng/ml for 200 mg and 500 mg groups, respectively. Cₘₐₓ values after a single 900 mg dose is 293+/−63.8 ng/ml. The Cₘᵢₙ values also increase proportionally with dose from a value of 2.34 ng/ml (for a group administered 50 mg) to 23.79 and 56.71 ng/ml for 200 mg and 500 mg groups, respectively. Tₘₐₓ is from about 1.2 to about 2.5 hours after administration.

In embodiments, methods of treating a developmental disorder and/or a seizure disorder are provided which include administering to a patient in need thereof a pharmaceutical composition including flupirtine or a pharmaceutical salt thereof, and ganaxolone. In embodiments, methods of treating a developmental disorder and/or a seizure disorder are provided which include administering to a patient in need thereof a pharmaceutical composition including flupirtine or a pharmaceutical salt thereof, and ganaxolone wherein the composition provides improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and a pharmaceutical composition including ganaxolone. In embodiments, methods of treating a developmental disorder and/or a seizure disorder are provided which include administering to a patient in need thereof a pharmaceutical composition including flupirtine or a pharmaceutical salt thereof, and a pharmaceutical composition including ganaxolone wherein the compositions provide improvement in one or more symptoms of the disorder.

In embodiments, methods of treating a developmental disorder and/or a seizure disorder are provided which include administering to a patient in need thereof a pharmaceutical composition including flupirtine or a pharmaceutical salt thereof, and ganaxolone wherein the composition provides improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutical salt thereof, and a pharmaceutical composition including ganaxolone wherein the compositions provide improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and ganaxolone wherein the composition provides improvement in one or more symptoms of the disorder for more than 8, 10, 12, 14, 16, 18, 20, 22 or 24 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and a pharmaceutical composition including ganaxolone wherein the compositions provide improvement in one or more symptoms of the disorder for more than 8, 10, 12, 14, 16, 18, 20, 22 or 24 hours after administration to the patient.

In embodiments, flupirtine or a pharmaceutically acceptable salt thereof, and ganaxolone may be administered in separate dosage forms or combined in one dosage form. In embodiments, flupirtine or a pharmaceutically acceptable salt thereof may be co-administered simultaneously with ganaxolone or a pharmaceutically acceptable salt thereof, or at spaced apart intervals.

In embodiments, methods of treating a developmental disorder and/or a seizure disorder include administering to a patient in need thereof any of the previously recited amounts of flupirtine or a pharmaceutically acceptable salt thereof, and about 0.5 mg to about 2000 mg of ganaxolone. In embodiments, methods of treating a developmental disorder and/or a seizure disorder include administering to a patient in need thereof flupirtine or a pharmaceutically acceptable salt thereof, and about 0.5 mg to about 2000 mg ganaxolone. In embodiments, any of the previously recited amounts of flupirtine or a pharmaceutically acceptable salt thereof and about 0.5 mg to about 2000 mg of ganaxolone is administered in 24 hours. In embodiments, any of the previously recited amounts of flupirtine or a pharmaceutically acceptable salt thereof and about 0.5 mg to about 2000 mg of ganaxolone is administered in divided doses over 24 hours.

In embodiments, ganaxolone can be administered at dosages ranging from about 0.01 mg/kg and about 20 mg/kg of body weight of a patient in need thereof, e.g., from about 0.1 mg/kg to 10 mg/kg at least once a day. In embodiments, an infusion dose may be administered at a rate of 1, 2, 3, 4, 5, 6, 7, 8, 9, or 10 mg/hr or in a range of about 1 mg/kg/hr to about 10 mg/kg/hr or 2 mg/kg/hr to about 8 mg/kg/hrs. In embodiments, ganaxolone can be administered in amounts between about 0.5 mg to about 2000 mg per day.

In embodiments, methods of treating a developmental disorder and/or a seizure disorder are provided which include administering flupirtine or a pharmaceutically acceptable salt thereof in any of the amounts discussed above to a patient in need thereof along with ganaxolone in an amount of about 0.5 mg to about 2000 mg. In embodiments, flupirtine or a pharmaceutically acceptable salt thereof and ganaxolone are administered to a patient in an amount which is effective to provide improvement in one or more symptoms of the disorders mentioned above. In embodiments, methods of treating a disorder described herein include administering flupirtine or a pharmaceutically acceptable salt thereof and ganaxolone to a patient in need thereof in an amount which is effective to provide improvement in next day functioning of the patient. In embodiments, the amount of ganaxolone administered daily can be between about 0.5 mg and 2000 mg. For example, the daily dosage can be 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, or 2000 mg ganaxolone. In embodiments, an adult dose can be about 500-1000 mg per day and can be increased to 1500 mg or 1800 mg per day. In embodiments, flupirtine or a pharmaceutically acceptable salt thereof and ganaxolone can be administered 2, 3 or 4 times daily in divided doses. In embodiments, the subject may be started at a low dose and the dosage is escalated. In this manner, it can be determined if the drugs are well tolerated in the subject. Dosages can be lower for infants and children than for adults. In embodiments, a ganaxolone dose in children can be about 100-1000 mg per day in 2, 3 or 4 divided doses.

In embodiments, methods of treating a developmental disorder and/or a seizure disorder are provided which include administering flupirtine or a pharmaceutically acceptable salt thereof in an amount discussed above and allopregnanolone to a patient in need thereof.

Allopregnanolone also known as 3α-hydroxy-5α-pregnan-20-one or 3α,5α-tetrahydroprogesterone (3α,5α-THP), is an endogenous inhibitory pregnane neurosteroid. It is synthesized from progesterone, and is a potent positive allosteric modulator of the action of gamma-aminobutyric acid (GABA) at GABA_{A} receptor. Allopregnanolone is reportedly under development as an intravenously administered drug for the treatment of super-refractory status epilepticus, postpartum depression, and essential tremor.

In embodiments, methods of treating a developmental disorder and/or a seizure disorder are provided which include administering to a patient in need thereof any of the previously recited amounts of flupirtine or a pharmaceutical salt thereof, and allopregnanolone. In embodiments, methods of treating a developmental disorder and/or a seizure disorder are provided which include administering to a patient in need thereof a pharmaceutical composition including any of the previously recited amounts of flupirtine or a pharmaceutical salt thereof, and allopregnanolone. In embodiments, methods of treating a developmental disorder and/or a seizure disorder are provided which include administering to a patient in need thereof a pharmaceutical composition including any of the previously recited amounts of flupirtine or a pharmaceutical salt thereof, and a pharmaceutical composition including allopregnanolone. In embodiments, about 0.005 mg to about 2000 mg of allopregnanolone are administered to a patient.

In embodiments, methods of treating a developmental disorder and/or a seizure disorder are provided which include administering to a patient in need thereof flupirtine or a pharmaceutical salt thereof, and allopregnanolone wherein the patient exhibits improvement in one or more symptoms of the disorder. In embodiments, methods of treating a developmental disorder and/or a seizure disorder are provided which include administering to a patient in need thereof flupirtine or a pharmaceutical salt thereof, and allopregnanolone in an amount effective to provide the patient with improvement in one or more symptoms of the disorder. In embodiments, methods of treating a developmental disorder and/or a seizure disorder are provided which include administering to a patient in need thereof flupirtine or a pharmaceutical salt thereof, and allopregnanolone wherein the patient exhibits improvement in one or more symptoms of the disorder for more than 6 hours after administration to the patient. In embodiments, methods of treating a developmental disorder and/or a seizure disorder are provided which include administering to a patient in need thereof flupirtine or a pharmaceutical salt thereof, and allopregnanolone in an amount effective to provide the patient with improvement in one or more symptoms of the disorder for more than 6 hours after administration to the patient. In embodiments, methods of treating a developmental disorder and/or a seizure disorder are provided which include administering to a patient in need thereof flupirtine or a pharmaceutically acceptable salt thereof, and allopregnanolone wherein improvement in one or more symptoms of the disorder occurs for more than 8, 10, 12, 14, 16, 18, 20, 22 or 24 hours after administration to the patient. In embodiments, methods of treating a developmental disorder and/or a seizure disorder are provided which include administering to a patient in need thereof flupirtine or a pharmaceutically acceptable salt thereof, and allopregnanolone in an amount effective to provide the patient with improvement in one or more symptoms of the disorder occurs for more than 8, 10, 12, 14, 16, 18, 20, 22 or 24 hours after administration to the patient. In embodiments, methods of treating a developmental disorder and/or a seizure disorder are provided which include administering to a patient in need thereof flupirtine or a pharmaceutical salt thereof, and allopregnanolone wherein the patient exhibits improvement in next day functioning of the patient. In embodiments, methods of treating a developmental disorder and/or a seizure disorder are provided which include administering to a patient in need thereof flupirtine or a pharmaceutical salt thereof, and allopregnanolone in an amount effective to provide the patient with improvement in next day functioning of the patient. In embodiments, any of the previously recited amounts of flupirtine or a pharmaceutical salt thereof, and about 0.005 mg to about 2000 mg of allopregnanolone are administered to a patient.

In embodiments, methods of treating a developmental disorder and/or a seizure disorder are provided which include administering to a patient in need thereof a pharmaceutical composition including flupirtine or a pharmaceutical salt thereof, and allopregnanolone wherein the composition provides improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and a pharmaceutical composition including allopregnanolone wherein the compositions provide improvement in one or more symptoms of the disorder. In embodiments, methods of treating a developmental disorder and/or a seizure disorder are provided which include administering to a patient in need thereof a pharmaceutical composition including flupirtine or a pharmaceutical salt thereof, and allopregnanolone wherein the composition provides improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutical salt thereof, and a pharmaceutical composition including allopregnanolone wherein the compositions provide improvement in one or more symptoms of the disorder for more than 6 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and ganaxolone wherein the composition provides improvement in one or more symptoms of the disorder for more than 8, 10, 12, 14, 16, 18, 20, 22 or 24 hours after administration to the patient. In embodiments, methods of treating a developmental disorder and/or a seizure disorder are provided which include administering to a patient in need thereof a pharmaceutical composition including flupirtine or a pharmaceutically acceptable salt thereof, and a pharmaceutical composition including ganaxolone wherein the compositions provide improvement in one or more symptoms of the disorder for more than 8, 10, 12, 14, 16, 18, 20, 22 or 24 hours after administration to the patient.

In embodiments, methods of treating a developmental disorder and/or a seizure disorder are provided which include administering to a patient in need thereof a pharmaceutical composition including flupirtine or a pharmaceutical salt thereof, and allopregnanolone wherein the composition provides the patient with an improvement in next day functioning. In embodiments, methods of treating a developmental disorder and/or a seizure disorder are provided which include administering to a patient in need thereof a pharmaceutical composition including flupirtine or a pharmaceutical salt thereof, and a pharmaceutical composition including allopregnanolone wherein the compositions provide the patient with an improvement in next day functioning.

In embodiments, flupirtine or a pharmaceutically acceptable salt thereof, and allopregnanolone or a pharmaceutically acceptable salt thereof may be administered in separate dosage forms or combined in one dosage form. In embodiments, flupirtine or a pharmaceutically acceptable salt thereof may be co-administered simultaneously with allopregnanolone or a pharmaceutically acceptable salt thereof, or at spaced apart intervals.

In embodiments, methods of treating a developmental disorder and/or a seizure disorder include administering to a patient in need thereof any of the previously recited amounts of flupirtine or a pharmaceutically acceptable salt thereof, and about 0.005 mg to about 2000 mg of allopregnanolone. In embodiments, methods of treating a developmental disorder and/or a seizure disorder include administering to a patient in need thereof flupirtine or a pharmaceutically acceptable salt thereof, and about 0.005 mg to about 2000 mg allopregnanolone. In embodiments, any of the previously recited amounts of flupirtine or a pharmaceutically acceptable salt thereof and about 0.005 mg to about 2000 mg of allopregnanolone is administered in 24 hours. In embodiments, any of the previously recited amounts of flupirtine or a pharmaceutically acceptable salt thereof and about 0.005 mg to about 2000 mg of allopregnanolone is administered in divided doses over 24 hours.

In embodiments, allopregnanolone can be administered at dosages ranging from about 0.001 mg/kg and about 1000 mg/kg of body weight of a patient in need thereof, e.g., from about 0.015 mg/kg to 500 mg/kg, about 0.05 mg/kg to about 500 mg/kg, about 0.1 mg/kg to about 500 mg/kg, about 0.5 mg/kg to about 100 mg/kg, about 0.75 mg/kg to about 75 mg/kg, about 1 mg/kg to about 50 mg/kg, or about 5 mg/kg to about 50 mg/kg, at least once a day. In embodiments, dosages can be 0.01, 0.015, 0.025, 0.03, 0.045, 0.05, 0.075, 0.1, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, or 10 mg. In embodiments, an infusion dose may be administered e.g., at a rate of 0.01, 0.015, 0.025, 0.03, 0.045, 0.05, 0.075, 0.1, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, or 10 mg/hr, or multiples thereof, or, e.g., in a range of about 0.01 mg/kg/hr to about 10 mg/kg/hr or 0.02 mg/kg/hr to about 8 mg/kg/hr.

In embodiments, methods of treating a developmental disorder and/or a seizure disorder are provided which include administering flupirtine or a pharmaceutically acceptable salt thereof in any of the amounts discussed above to a patient in need thereof along with allopregnanolone in an amount of about 0.005 mg to about 2000 mg. In embodiments, flupirtine or a pharmaceutically acceptable salt thereof and allopregnanolone are administered to a patient in an amount which is effective to provide improvement in one or more symptoms of the disorders mentioned above. In embodiments, methods of treating a disorder described herein include administering flupirtine or a pharmaceutically acceptable salt thereof and allopregnanolone to a patient in need thereof in an amount which is effective to provide improvement in next day functioning of the patient. In embodiments, the amount of allopregnanolone administered daily can be between about 0.005 mg and 2000 mg. For example, the daily dosage can be 0.005 mg, 0.01 mg, 0.025 mg, 0.05 mg, 0.075 mg, 0.1 mg, 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 11 mg, 12 mg, 13 mg, 14 mg, 15 mg, 16 mg, 17 mg, 18 mg, 19 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, or 2000 mg allopregnanolone. In embodiments, an adult dose can be about 1-1000 mg per day and can be increased to 1500 mg or 1800 mg per day. In embodiments, flupirtine or a pharmaceutically acceptable salt thereof and allopregnanolone can be administered 2, 3 or 4 times daily in divided doses. In embodiments, the subject may be started at a low dose and the dosage is escalated. In this manner, it can be determined if the drugs are well tolerated in the subject. Dosages can be lower for infants and children than for adults. In embodiments, a allopregnanolone dose in children can be, e.g., about 0.05-1000 mg per day in 2, 3 or 4 divided doses.

In embodiments, provided herein are methods of treating a developmental disorder and/or seizure disorder including administering to a patient in need thereof a pharmaceutical composition including flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, wherein the patient exhibits improvement in at least one symptom of the developmental disorder and/or seizure disorder. Symptoms may include, but are not limited to, ataxia, gait, speech impairment, vocalization, cognition, motor activity, clinical seizure, subclinical seizure, hypotonia, hypertonia, feeding difficulty, drooling, mouthing behavior, sleep difficulties, hand flapping, hand ringing, teeth grinding, easily provoked laughter and short attention span. In embodiments, provided in accordance with the present disclosure is improvement in cognition. Cognition refers to the mental processes involved in gaining knowledge and comprehension, such as thinking, knowing, remembering, judging, and problem solving. These higher-level functions of the brain encompass language, imagination, perception, and the planning and execution of complex behaviors. The precise dosage will vary according to a variety of factors such as subject-dependent variables (e.g., age, immune system health, clinical symptoms etc.).

In embodiments, the methods and compositions provided may reduce or prevent one or more different types of seizures. Generally, a seizure can include convulsions, repetitive movements, unusual sensations, and combinations thereof. Seizures can be categorized as focal seizures (also referred to as partial seizures) and generalized seizures. Focal seizures affect only one side of the brain, while generalized seizures affect both sides of the brain. Specific types of focal seizures include simple focal seizures, complex focal seizures, and secondarily generalized seizures. Simple focal seizures can be restricted or focused on a particular lobe (e.g., temporal lobe, frontal lobe, parietal lobe, or occipital lobe). Complex focal seizures generally affect a larger part of one hemisphere than simple focal seizures, but commonly originate in the temporal lobe or the frontal lobe. When a focal seizure spreads from one side (hemisphere) to both sides of the brain, the seizure is referred to as a secondarily generalized seizure. Specific types of generalized seizures include absences (also referred to as petit mal seizures), tonic seizures, atonic seizures, myoclonic seizures, tonic clonic seizures (also referred to as grand mal seizures), and clonic seizures.

Accordingly, provided herein are methods of treating a developmental disorder and/or seizure disorder including administering to a patient in need thereof a pharmaceutical composition including flupirtine or a pharmaceutically acceptable salt thereof, either alone or in combination with one or more of retigabine, N-[2-Amino-4-[[(2,4,6-trimethylphenyl)methyl]amino]phenyl]-carbamic acid-ethyl ester, gaboxadol, pipradrol, ganaxolone or allopregnanolone, or a pharmaceutically acceptable salt of any of the preceding, after a warning sign of an impending seizure is detected to reduce or prevent seizure activity. In embodiments, a continuing regimen of administration of active agents herein is effective to reduce or prevent occurrence of seizure activity.

In embodiments, the methods described herein are effective to reduce, delay, or prevent one or more other clinical symptoms of a developmental disorder and/or seizure disorder. For example, the effect of a composition including flupirtine or a pharmaceutically acceptable salt thereof, alone or in combination with one or more of retigabine, N-[2-Amino-4-[[(2,4,6-trimethylphenyl)methyl]-amino]phenyl]-carbamic acid-ethyl ester, gaboxadol, pipradrol, ganaxolone or allopregnanolone, or a pharmaceutically acceptable salt of any of the preceding, on a particular symptom, pharmacologic, or physiologic indicator can be compared to an untreated subject, or the condition of the subject prior to treatment. In embodiments, the symptom, pharmacologic, and/or physiologic indicator is measured in a subject prior to treatment, and again one or more times after treatment is initiated. In embodiments, the control is a reference level, or average determined based on measuring the symptom, pharmacologic, or physiologic indicator in one or more subjects that do not have the disease or condition to be treated (e.g., healthy subjects). In embodiments, the effect of the treatment is compared to a conventional treatment that is known the art.

Effective treatment of a developmental disorder and/or a seizure disorder (e.g., acute repetitive seizure, status epilepticus) herein may be established by showing reduction in the frequency or severity of symptoms (e.g., more than 10%, 20%, 30% 40% or 50%) after a period of time compared with baseline. For example, after a baseline period of 1 month, the patients may be randomly allocated flupirtine, either alone or with one or more of retigabine, N-[2-Amino-4-[[(2,4,6-trimethylphenyl)-methyl]amino]phenyl]-carbamic acid ethyl ester, gaboxadol, pipradrol, ganaxolone, allopregnanolone, and a pharmaceutically acceptable salt of any of the preceding, or placebo as add-on therapy to standard therapies, during a double-blind period of 2 months. Primary outcome measurements may include the percentage of responders on flupirtine, either alone or with one or more of retigabine, N-[2-Amino-4-[[(2,4,6-trimethylphenyl)-methyl]amino]phenyl]-carbamic acid ethyl ester, gaboxadol, pipradrol, ganaxolone or allopregnanolone, or a pharmaceutically acceptable salt of any of the preceding, and on placebo, defined as having experienced at least a 10% to 50% reduction of symptoms during the second month of the double-blind period compared with baseline.

In embodiments, provided herein are methods of treating a developmental disorder and/or seizure disorder include administering to a patient in need thereof a pharmaceutical composition including flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, wherein the composition provides improvement of at least one symptom for more than 4 hours after administration of the pharmaceutical composition to the patient. In embodiments, the improvement of at least one symptom for more than 6 hours after administration of the pharmaceutical composition to the patient is provided in accordance with the present disclosure. In embodiments, improvement in at least one symptom for at least e.g., 8 hours, 10 hours, 12 hours, 15 hours, 18 hours, 20 hours, or 24 hours after administration of the pharmaceutical composition to the patient is provided in accordance with the present disclosure. In embodiments, improvement in at least one symptom for 12 hours after administration of the pharmaceutical composition to the patient is provided in accordance with the present disclosure.

In embodiments, provided herein are methods of treating a developmental disorder and/or seizure disorder including administering to a patient in need thereof a pharmaceutical composition including flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, wherein the composition provides improvement in next day functioning to the patient.

In embodiments, provided herein are methods of treating a developmental disorder and/or seizure disorder including administering to a patient in need thereof flupirtine or a pharmaceutically acceptable salt thereof, alone or in combination with one or more of retigabine, N-[2-Amino-4-[[(2,4,6-trimethylphenyl)methyl]-amino]phenyl]-carbamic acid-ethyl ester, gaboxadol, pipradrol, ganaxolone or allopregnanolone, or a pharmaceutically acceptable salt of any of the preceding, which provides an in vivo plasma profile, wherein the in vivo plasma profile of the patient 10 hours after administration of flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, is reduced by more than 50% and the method provides improvement in the patient for more than 10, 12, 14, 16, 18, 20, 22 or 24 hours after administration. In embodiments, provided herein are methods of treating a developmental disorder and/or seizure disorder including administering to a patient in need thereof flupirtine or a pharmaceutically acceptable salt thereof, alone or in combination with one or more of retigabine, N-[2-Amino-4-[[(2,4,6-trimethylphenyl)methyl]-amino]phenyl]-carbamic acid-ethyl ester, gaboxadol, pipradrol, ganaxolone or allopregnanolone, or a pharmaceutically acceptable salt of any of the preceding, which provides an in vivo plasma profile, wherein the in vivo plasma profile of the patient 10 hours after administration of flupirtine or a pharmaceutically acceptable salt thereof, alone or in combination with one or more of retigabine, N-[2-Amino-4-[[(2,4,6-trimethylphenyl)methyl]-amino]phenyl]carbamic acid-ethyl ester, gaboxadol, pipradrol, ganaxolone or allopregnanolone, or a pharmaceutically acceptable salt of any of the preceding, is reduced by more than 55% and the method provides improvement in the patient for more than 10, 12, 14, 16, 18, 20, 22 or 24 hours after administration. In embodiments, provided herein are methods of treating a developmental disorder and/or seizure disorder including administering to a patient in need thereof flupirtine or a pharmaceutically acceptable salt thereof, alone or in combination with one or more of retigabine, N-[2-Amino-4-[[(2,4,6-trimethylphenyl)methyl]amino]phenyl]-carbamic acid-ethyl ester, gaboxadol, pipradrol, ganaxolone or allopregnanolone, or a pharmaceutically acceptable salt of any of the preceding, which provides an in vivo plasma profile, wherein the in vivo plasma profile of the patient 10 hours after administration flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, is reduced by more than 60% and the method provides improvement in the patient for more than 10, 12, 14, 16, 18, 20, 22 or 24 hours after administration. In embodiments, provided herein are methods of treating a developmental disorder and/or seizure disorder including administering to a patient in need thereof flupirtine or a pharmaceutically acceptable salt thereof, alone or in combination with one or more of retigabine, N-[2-Amino-4-[[(2,4,6-trimethylphenyl)methyl]amino]phenyl]-carbamic acid-ethyl ester, gaboxadol, pipradrol, ganaxolone or allopregnanolone, or a pharmaceutically acceptable salt of any of the preceding, which provides an in vivo plasma profile, wherein the in vivo plasma profile of the patient 10 hours after administration of flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, is reduced by more than 65% and the method provides improvement in the patient for more than 6, 8, 10, 12, 14, 16, 18, 20, 22 or 24 hours after administration. In embodiments, provided herein are methods of treating a developmental disorder and/or seizure disorder including administering to a patient in need thereof flupirtine or a pharmaceutically acceptable salt thereof, alone or in combination with one or more of retigabine, N-[2-Amino-4-[[(2,4,6-trimethylphenyl)methyl]amino]phenyl]carbamic acid-ethyl ester, gaboxadol, pipradrol, ganaxolone or allopregnanolone, or a pharmaceutically acceptable salt of any of the preceding, which provides an in vivo plasma profile, wherein the in vivo plasma profile of the patient 10 hours after administration of flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, is reduced by more than 70% and the method provides improvement in the patient for more than 6, 8, 10, 12, 14, 16, 18, 20, 22 or 24 hours after administration.

In embodiments, provided herein are methods of treating developmental disorder or seizure disorder wherein the amount of active substance, e.g., flupirtine or a pharmaceutically acceptable salt thereof, alone or in combination with one or more of retigabine, N-[2-Amino-4-[[(2,4,6-trimethylphenyl)methyl]amino]phenyl]-carbamic acid-ethyl ester, gaboxadol, pipradrol, ganaxolone or allopregnanolone, or a pharmaceutically acceptable salt of any of the preceding, within the patient about 4 hours after administration of the pharmaceutical composition is less than about 75% of the administered dose. In embodiments, provided herein are methods wherein the amount of flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, within the patient about, e.g., 6 hours, 8 hours, 10 hours, 12 hours, 15 hours, or 20 hours after administration of the pharmaceutical composition is less than about 75%.

In embodiments, provided herein are methods of treating a developmental disorder wherein the amount of active substance, e.g., flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, within the patient about 4 hours after administration of the pharmaceutical composition is less than about 80% of the administered dose. In embodiments, provided herein are methods wherein the amount of active substance, e.g., flupirtine or a pharmaceutically acceptable salt thereof, alone or in combination with one or more of retigabine, N-[2-Amino-4-[[(2,4,6-trimethylphenyl)methyl]-amino]phenyl]carbamic acid-ethyl ester, gaboxadol, pipradrol, ganaxolone or allopregnanolone, or a pharmaceutically acceptable salt of any of the preceding, within the patient about, e.g., 6 hours, 8 hours, 10 hours, 12 hours, 15 hours, or 20 hours after administration of the pharmaceutical composition is less than about 80% of the administered dose.

In embodiments, provided herein are methods of treating developmental disorder wherein the amount of active substance, e.g., flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, within the patient about 4 hours after administration of the pharmaceutical composition is between about 65% to about 85% of the administered dose. In embodiments, the amount of active substance, e.g., flupirtine or a pharmaceutically acceptable salt thereof, alone or in combination with one or more of retigabine, N-[2-Amino-4-[[(2,4,6-trimethylphenyl)methyl]amino]-phenyl]-carbamic acid-ethyl ester, gaboxadol, pipradrol, ganaxolone or allopregnanolone, or a pharmaceutically acceptable salt of any of the preceding, within the patient after about, e.g., 6 hours, 8 hours, 10 hours, 12 hours, 15 hours, or 20 hours after administration of the pharmaceutical composition is between about 65% to about 85% of the administered dose.

In embodiments, the pharmaceutical compositions described herein may be administered once daily, twice daily, three times daily, four times daily, or every other day. In embodiments, the pharmaceutical compositions described herein may be administered by continuous infusion. In embodiments, a pharmaceutical composition described herein is provided to the patient in the morning. In embodiments, a pharmaceutical composition described herein is provided to the patient in the evening. In embodiments, a pharmaceutical composition described herein is provided to the patient once in the evening and once in the morning. In embodiments, a pharmaceutical composition described herein is provided to the patient once in the morning, once in the afternoon and once in the evening.

In embodiments, as mentioned previously, pharmaceutical compositions herein may be provided with conventional release or modified release profiles. Pharmaceutical compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers.

In embodiments, pharmaceutical compositions herein are modified release dosage forms which provide modified release profiles. Modified release profiles may exhibit immediate release, delayed release, or extended release profiles. Conventional (or unmodified) release oral dosage forms such as tablets, capsules, suppositories, syrups, solutions and suspensions typically release medications into the mouth, stomach or intestines as the tablet, capsule shell or suppository dissolves, or, in the case of syrups, solutions and suspensions, when they are swallowed. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Patients with developmental disorders, e.g., Angelman syndrome, Fragile X syndrome, Fragile X-associated tremor/ataxia syndrome, Rett syndrome, and/or seizure disorders may exhibit such behavior. ODDF's can provide rapid delivery of medication to the blood stream through mucosa resulting in a rapid onset of action. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which one or more of flupirtine, retigabine, N-[2-Amino-4-[[(2,4,6-trimethylphenyl)methyl]amino]phenyl]carbamic acid-ethyl ester, gaboxadol, pipradrol, ganaxolone or allopregnanolone, or a pharmaceutically acceptable salt of any of the preceding, is applied to beads, e.g., confectioners nonpareil beads, and then coated with conventional release retarding materials such as waxes, enteric coatings and the like. In embodiments, beads can be formed in which one or more of flupirtine, retigabine, N-[2-Amino-4-[[(2,4,6-trimethylphenyl)methyl]amino]phenyl]carbamic acid-ethyl ester, gaboxadol, pipradrol, ganaxolone or allopregnanolone, or a pharmaceutically acceptable salt of any of the preceding, is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as one or more of flupirtine, retigabine, N-[2-Amino-4-[[(2,4,6-trimethylphenyl)methyl]amino]-phenyl]carbamic acid-ethyl ester, gaboxadol, pipradrol, ganaxolone or allopregnanolone, or a pharmaceutically acceptable salt of any of the preceding, at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

Delayed release dosage forms are known to those skilled in the art. For example, coated delayed release beads or granules in which one or more of flupirtine, retigabine, N-[2-Amino-4-[[(2,4,6-trimethylphenyl)methyl]amino]-phenyl]-carbamic acid ethyl ester, gaboxadol, pipradrol, ganaxolone or allopregnanolone, or a pharmaceutically acceptable salt of any of the preceding, is applied to beads, e.g., confectioners nonpareil beads, and then coated with conventional release delaying materials such as waxes, enteric coatings and the like. In embodiments, beads can be formed in which one or more of flupirtine, retigabine, N-[2-Amino-4-[[(2,4,6-trimethylphenyl)methyl]amino]phenyl]-carbamic acid-ethyl ester, gaboxadol, pipradrol, ganaxolone or allopregnanolone, or a pharmaceutically acceptable salt of any of the preceding, is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of one or more of flupirtine, retigabine, N-[2-Amino-4-[[(2,4,6-trimethylphenyl)methyl]amino]-phenyl]carbamic acid-ethyl ester, gaboxadol, pipradrol, ganaxolone or allopregnanolone, or a pharmaceutically acceptable salt of any of the preceding, can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, one or more of flupirtine, retigabine, N-[2-Amino-4-[[(2,4,6-trimethylphenyl)methyl]amino]phenyl]carbamic acid-ethyl ester, gaboxadol, pipradrol, ganaxolone or allopregnanolone, or a pharmaceutically acceptable salt of any of the preceding, is incorporated into porous inert carriers that provide delayed release profiles. In embodiments, the porous inert carriers incorporate channels or passages from which the drug diffuses into surrounding fluids. In embodiments, one or more of flupirtine, retigabine, N-[2-Amino-4-[[(2,4,6-trimethylphenyl)-methyl]amino]phenyl]carbamic acid-ethyl ester, gaboxadol, pipradrol, ganaxolone or allopregnanolone, or a pharmaceutically acceptable salt of any of the preceding, is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, pharmaceutical compositions described herein are suitable for parenteral administration, including, e.g., intramuscular (i.m.), intravenous (i.v.), subcutaneous (s.c.), intraperitoneal (i.p.), or intrathecal (i.t.). Parenteral compositions must be sterile for administration by injection, infusion or implantation into the body and may be packaged in either single-dose or multi-dose containers. In embodiments, liquid pharmaceutical compositions for parenteral administration to a subject include an active substance, e.g., flupirtine or a pharmaceutically acceptable salt thereof alone or in combination with one or more of retigabine, N-[2-Amino-4-[[(2,4,6-trimethylphenyl)methyl]-amino]phenyl]carbamic acid ethyl ester, gaboxadol, pipradrol, ganaxolone or allopregnanolone, or a pharmaceutically acceptable salt of any of the preceding, in any of the respective amounts described above. In embodiments, the liquid pharmaceutical compositions include an active substance, e.g., flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding, at a respective concentration of, e.g., about 0.05 μg/ml to about 50 μg/ml, about 0.1 μg/ml to about 50 μg/ml, about 0.05 μg/ml to about 25 μg/ml, about 0.05 μg/ml to about 10 μg/ml, about 0.05 μg/ml to about 5 μg/ml, or about 0.05 μg/ml to about 1 μg/ml. In embodiments, the pharmaceutical composition includes an active substance, e.g., flupirtine or a pharmaceutically acceptable salt thereof alone or in combination with one or more of retigabine, N-[2-Amino-4-[[(2,4,6-trimethylphenyl)methyl]-amino]phenyl]carbamic acid ethyl ester, gaboxadol, pipradrol, ganaxolone or allopregnanolone, or a pharmaceutically acceptable salt of any of the preceding, at a respective concentration of, e.g., about 0.05 μg/ml to about 15 μg/ml, about 0.5 μg/ml to about 10 μg/ml, about 0.5 μg/ml to about 7 μg/ml, about 1 μg/ml to about 10 μg/ml, about 5 μg/ml to about 10 μg/ml, or about 5 μg/ml to about 15 μg/ml. In embodiments, the pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are contained in a bag, a glass vial, a plastic vial, or a bottle.

In embodiments, pharmaceutical compositions for parenteral administration include about 0.05 mg to about 100 mg active substance, e.g., flupirtine or a pharmaceutically acceptable salt thereof alone or in combination with one or more of retigabine, N-[2-Amino-4-[[(2,4,6-trimethylphenyl)methyl]amino]phenyl]-carbamic acid ethyl ester, gaboxadol, pipradrol, ganaxolone or allopregnanolone, or a pharmaceutically acceptable salt of any of the preceding. In embodiments, the pharmaceutical compositions include about, e.g., 0.1 mg to 25 mg, 0.1 mg to 20 mg, 0.1 mg to 15 mg, 0.5 mg to 25 mg, 0.5 mg to 20 mg, 0.5 to 15 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 25 mg, 2.5 mg to 20 mg, 2.5 mg to 15 mg, 3 mg to 25 mg, 3 mg to 20 mg, 3 mg to 15 mg active substance, e.g., flupirtine or a pharmaceutically acceptable salt thereof alone or in combination with one or more of retigabine, N-[2-Amino-4-[[(2,4,6-trimethylphenyl)methyl]-amino]phenyl]carbamic acid ethyl ester, gaboxadol, pipradrol, ganaxolone or allopregnanolone, or a pharmaceutically acceptable salt of any of the preceding, respectively.

In embodiments, pharmaceutical compositions for parenteral administration to a subject include an active substance, e.g., flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding, at a respective concentration of about 0.005 mg/ml to about 500 mg/ml. In embodiments, the pharmaceutical composition for parenteral administration includes an active substance, e.g., flupirtine or a pharmaceutically acceptable salt thereof alone or in combination with one or more of retigabine, N-[2-Amino-4-[[(2,4,6-trimethylphenyl)-methyl]amino]phenyl]carbamic acid ethyl ester, gaboxadol, pipradrol, ganaxolone or allopregnanolone, or a pharmaceutically acceptable salt of any of the preceding, at a respective concentration of, e.g., about 0.05 mg/ml to about 50 mg/ml, about 0.1 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the pharmaceutical composition for parenteral administration includes an active substance, e.g., flupirtine or a pharmaceutically acceptable salt thereof alone or in combination with one or more of retigabine, N-[2-Amino-4-[[(2,4,6-trimethylphenyl)-methyl]amino]phenyl]carbamic acid ethyl ester, gaboxadol, pipradrol, ganaxolone or allopregnanolone, or a pharmaceutically acceptable salt of any of the preceding, at a respective concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml. In embodiments, pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are packages and stored in a bag, a glass vial, a plastic vial, or a bottle.

In embodiments, pharmaceutical compositions for parenteral administration include an active substance, e.g., flupirtine or a pharmaceutically acceptable salt thereof alone or in combination with one or more of retigabine, N-[2-Amino-4-[[(2,4,6-trimethylphenyl)methyl]amino]phenyl]carbamic acid ethyl ester, gaboxadol, pipradrol, ganaxolone or allopregnanolone, or a pharmaceutically acceptable salt of any of the preceding, wherein the active substance is present at a molarity less than about 1.0 M. In embodiments, the active substance, e.g., flupirtine or a pharmaceutically acceptable salt thereof alone or in combination with one or more of retigabine, N-[2-Amino-4-[[(2,4,6-trimethylphenyl)methyl]amino]phenyl]-carbamic acid ethyl ester, gaboxadol, pipradrol, ganaxolone or allopregnanolone, or a pharmaceutically acceptable salt of any of the preceding, is present at a molarity greater than, e.g., about 0.0001 M about 0.001 M, about 0.01 M, about 0.1 M, about 0.2 M, greater than about 0.5, greater than about 1.0 M, greater than about 1.2 M, greater than about 1.5 M, greater than about 1.75 M, greater than about 2.0 M, or greater than about 2.5 M. In embodiments, the active substance, e.g., flupirtine or a pharmaceutically acceptable salt thereof alone or in combination with one or more of retigabine, N-[2-Amino-4-[[(2,4,6-trimethylphenyl)methyl]-amino]phenyl]carbamic acid ethyl ester, gaboxadol, pipradrol, ganaxolone or allopregnanolone, or a pharmaceutically acceptable salt of any of the preceding, is present at a molarity of between, e.g., about 0.00001 M to about 0.1 M, about 0.01 to about 0.1 M, about 0.1 M to about 1.0 M, about 1.0 M to about 5.0 M, or about 5.0 M to about 10.0 M. In embodiments, the active substance, e.g., flupirtine or a pharmaceutically acceptable salt thereof alone or in combination with one or more of retigabine, N-[2-Amino-4-[[(2,4,6-trimethylphenyl)methyl]-amino]phenyl]carbamic acid ethyl ester, gaboxadol, pipradrol, ganaxolone or allopregnanolone, or a pharmaceutically acceptable salt of any of the preceding, is present at a molarity of less than, e.g., about 0.01 M, about 0.1 M, about 1.0 M, about 5.0 M, or about 10.0 M.

In embodiments, the solubility of the active substance, e.g., flupirtine or a pharmaceutically acceptable salt thereof alone or in combination with one or more of retigabine, N-[2-Amino-4-[[(2,4,6-trimethylphenyl)methyl]-amino]phenyl]carbamic acid ethyl ester, gaboxadol, pipradrol, ganaxolone or allopregnanolone, or a pharmaceutically acceptable salt of any of the preceding, in the pharmaceutical composition for parenteral administration is greater than, e.g., about 10 mg/mL, about 15 mg/mL, about 20 mg/mL, about 25 mg/mL, about 30 mg/mL, about 40 mg/mL, about 50 mg/mL, about 75 mg/mL, about 100 mg/mL, about 150 mg/mL, when measured, for example, in water at 25° C.

In embodiments, the solubility of the active substance, e.g., flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding, in the composition is between, e.g., about 1 mg/mL to about 50 mg/mL, about 5 mg/mL to about 50 mg/mL, about 10 mg/mL to about 50 mg/mL, about 20 mg/mL to about 50 mg/ml, from about 20 mg/mL to about 30 mg/mL or from about 10 mg/mL to about 45 mg/mL, when measured, for example, in water at 25 C.

In embodiments, a pharmaceutical composition for parenteral administration is provided wherein the pharmaceutical composition is stable for at least six months. In embodiments, the pharmaceutical compositions for parenteral administration exhibit no more than about 5% decrease in active substance, e.g., flupirtine or a pharmaceutically acceptable salt thereof alone or in combination with one or more of retigabine, N-[2-Amino-4-[[(2,4,6-trimethylphenyl)methyl]amino]phenyl]-carbamic acid ethyl ester, gaboxadol, pipradrol, ganaxolone or allopregnanolone, or a pharmaceutically acceptable salt of any of the preceding, e.g., 3 months or 6 months. In embodiments, the amount of flupirtine or a pharmaceutically acceptable salt thereof alone or in combination with one or more of retigabine, N-[2-Amino-4-[[(2,4,6-trimethylphenyl)methyl]-amino]phenyl]-carbamic acid ethyl ester, gaboxadol, pipradrol, ganaxolone or allopregnanolone, or a pharmaceutically acceptable salt of any of the preceding, degrades at no more than about, e.g., 2.5%, 1%, 0.5% or 0.1%. In embodiments, the degradation is less than about, e.g., 5%, 2.5%, 1%, 0.5%, 0.25%, 0.1%, for at least six months.

In embodiments, pharmaceutical compositions for parenteral administration are provided wherein the pharmaceutical composition remains soluble. In embodiments, pharmaceutical compositions for parenteral administration are provided that are stable, soluble, local site compatible and/or ready-to-use. In embodiments, the pharmaceutical compositions herein are ready-to-use for direct administration to a patient in need thereof. In embodiments, parenteral compositions containing allopregnanolone herein may include solubility enhancers such as a cyclodextrin. Examples of a cyclodextrin include, but are not limited to, hydroxypropyl-beta-cyclodextrin, sulfobutylether-beta-cyclodextrin sodium salt, or a mixture thereof.

The parenteral compositions provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding used in the composition. Thus, parenteral compositions are provided wherein there is no incompatibility between any of the components of the dosage form.

In embodiments, parenteral compositions including flupirtine or a pharmaceutically acceptable salt thereof alone or in combination with one or more of retigabine, N-[2-Amino-4-[[(2,4,6-trimethylphenyl)methyl]amino]-phenyl]-carbamic acid ethyl ester, gaboxadol, pipradrol, ganaxolone or allopregnanolone, or a pharmaceutically acceptable salt of any of the preceding include a stabilizing amount of at least one excipient. For example, excipients may be selected from the group consisting of buffering agents, solubilizing agents, tonicity agents, antioxidants, chelating agents, antimicrobial agents, and preservative. One skilled in the art will appreciate that an excipient may have more than one function and be classified in one or more defined group.

In embodiments, parenteral compositions including flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding, and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions may be administered as needed, e.g., once, twice, thrice or four or more times daily, or continuously depending on the patient's needs.

In embodiments, parenteral compositions of an active substance, e.g., flupirtine or a pharmaceutically acceptable salt thereof alone or in combination with one or more of retigabine, N-[2-Amino-4-[[(2,4,6-trimethylphenyl)methyl]-amino]phenyl]carbamic acid ethyl ester, gaboxadol, pipradrol, ganaxolone or allopregnanolone, or a pharmaceutically acceptable salt of any of the preceding are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

In embodiments, provided herein are methods of treating a developmental disorder and/or seizure disorder including administering to a patient in need thereof a pharmaceutical composition including an active substance, e.g., flupirtine or a pharmaceutically acceptable salt thereof, alone or in combination with one or more of retigabine, N-[2-Amino-4-[[(2,4,6-trimethylphenyl)-methyl]amino]phenyl]carbamic acid ethyl ester, gaboxadol, pipradrol, ganaxolone or allopregnanolone, or a pharmaceutically acceptable salt of any of the preceding, in a respective amount described herein, wherein the composition provides an in vivo plasma profile having a Cₘₐₓ less than about 800 ng/ml. In embodiments, the composition provides improvement for more than 6 hours after administration to the patient.

In embodiments, pharmaceutical compositions including flupirtine or a pharmaceutically acceptable salt thereof, alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding, provide an in vivo plasma profile having a Cₘₐₓ less than about, e.g., 2000 ng/ml, 1000 ng/ml, 850 ng/ml, 800 ng/ml, 750 ng/ml, 700 ng/ml, 650 ng/ml, 600 ng/ml, 550 ng/ml, 450 ng/ml, 400 ng/ml 350 ng/ml, or 300 ng/ml and wherein the composition provides improvement of next day functioning of the patient. In embodiments, the pharmaceutical composition provides an in vivo plasma profile having a Cₘₐₓ less than about, e.g., 250 ng/ml, 200 ng/ml 150 ng/ml, or 100 ng/ml and wherein the composition provides improvement of next day functioning of the patient. In embodiments, the pharmaceutical composition provides improvement in one or more symptoms of a disorder herein for more than 6 hours after administration.

In embodiments, provided herein are methods of treating a developmental disorder and/or a seizure disorder including administering to a patient in need thereof a pharmaceutical composition containing flupirtine or a pharmaceutically acceptable salt thereof, alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding, wherein the composition provides a consistent in vivo plasma profile having a AUC_{0-∞} of less than about 900 ng·hr/ml. In embodiments, the pharmaceutical composition provides improvement in next day functioning of the patient. In embodiments, the compositions provide an in vivo plasma profile having a AUC_{0-∞} of less than about, e.g., 850 ng·hr/ml, 800 ng·hr/ml, 750 ng·hr/ml, or 700 ng·hr/ml and wherein the pharmaceutical composition provides improvement of next day functioning of the patient. In embodiments, the composition provides improvement in one or more symptoms of a disorder herein for more than 6 hours after administration.

In embodiments, provided herein are methods of treating a developmental disorder and/or seizure disorder including administering to a patient in need thereof a pharmaceutical composition comprising an active substance, e.g., flupirtine or a pharmaceutically acceptable salt thereof, alone or in combination with one or more of retigabine, N-[2-Amino-4-[[(2,4,6-trimethylphenyl)-methyl]amino]phenyl]carbamic acid ethyl ester, gaboxadol, pipradrol, ganaxolone or allopregnanolone, or a pharmaceutically acceptable salt of any of the preceding, wherein the pharmaceutical composition provides an in vivo plasma profile having a AUC_{0-∞} of less than about, e.g., 650 ng·hr/ml, 600 ng·hr/ml, 550 ng·hr/ml, 500 ng·hr/ml, or 450 ng·hr/ml. In embodiments, the composition provides an in vivo plasma profile having a AUC_{0-∞} of less than about, e.g., 400 ng·hr/ml, 350 ng·hr/ml, 300 ng·hr/ml, 250 ng·hr/ml, or 200 ng·hr/ml. In embodiments, the pharmaceutical composition provides an in vivo plasma profile having a AUC_{0-∞} of less than about, e.g., 150 ng·hr/ml, 100 ng·hr/ml, 75 ng·hr/ml, or 50 ng·hr/ml. In embodiments, the pharmaceutical composition provides improvement of next day functioning of the patient after administration for more than, e.g., 4 hours, 6 hours, 8 hours, 10 hours, or 12 hours, after administration of the composition to the patient.

In embodiments, provided herein are methods of treating a developmental disorder and/or seizure disorder including administering to a patient in need thereof a first pharmaceutical composition including flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding, and a second pharmaceutical composition including flupirtine or a pharmaceutically acceptable salt thereof alone or in combination with one or more of retigabine, N-[2-Amino-4-[[(2,4,6-trimethylphenyl)-methyl]amino]-phenyl]carbamic acid ethyl ester, gaboxadol, pipradrol, ganaxolone or allopregnanolone, or a pharmaceutically acceptable salt of any of the preceding.

In embodiments, the second pharmaceutical composition provides an in vivo plasma profile having a mean AUC_{0-∞} which is about the same as the mean AUC_{0-∞} of the first pharmaceutical composition. In embodiments, the second pharmaceutical composition provides an in vivo plasma profile having a mean AUC_{0-∞} of at least about 20% less than the first pharmaceutical composition. In embodiments, provided herein are methods of treating a developmental disorder and/or seizure disorder including administering to a patient in need thereof a first pharmaceutical composition including flupirtine or a pharmaceutically acceptable salt thereof alone or in combination with one or more of retigabine, N-[2-Amino-4-[[(2,4,6-trimethylphenyl)methyl]amino]phenyl]carbamic acid ethyl ester, gaboxadol, pipradrol, ganaxolone or allopregnanolone, or a pharmaceutically acceptable salt of any of the preceding, and a second pharmaceutical composition including flupirtine or a pharmaceutically acceptable salt thereof alone or in combination with one or more of retigabine, N-[2-Amino-4-[[(2,4,6-trimethylphenyl)methyl]amino]phenyl]carbamic acid ethyl ester, gaboxadol, pipradrol, ganaxolone or allopregnanolone, or a pharmaceutically acceptable salt of any of the preceding, wherein the second pharmaceutical composition provides a stable in vivo plasma profile having a mean AUC_{0-∞} of at least about, e.g., 25%, 30%, 35%, 40%, 45% or 50% less than the first pharmaceutical composition. In embodiments, the compositions provide improvement of next day functioning of the patient. For example, the pharmaceutical compositions may provide improvement in one or more symptoms for more than about, e.g., 6 hours, 8 hours, 10 hours, 12 hours, 14 hours, 16 hours, 18 hours, 20 hours, 22 hours or 24 hours after administration of the first and/or second pharmaceutical composition.

In embodiments, provided herein are methods of treating a developmental disorder and/or seizure disorder including administering to a patient in need thereof a first pharmaceutical composition including flupirtine or a pharmaceutically acceptable salt thereof alone or in combination with at least one of one or more of retigabine, N-[2-Amino-4-[[(2,4,6-trimethylphenyl)-methyl]amino]-phenyl]carbamic acid ethyl ester, gaboxadol, pipradrol, ganaxolone or allopregnanolone, or a pharmaceutically acceptable salt of any of the preceding, and a second pharmaceutical composition including flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding, wherein the second pharmaceutical composition provides an in vivo plasma profile having a mean AUC_{0-∞} of less than about 900 ng·hr/ml. In embodiments, the second pharmaceutical composition provides an in vivo plasma profile having a AUC_{0-∞} of less than about, e.g., 800 ng·hr/ml, 750 ng·hr/ml, 700 ng·hr/ml, 650 ng·hr/ml, or 600 ng·hr/ml. In embodiments, the second pharmaceutical composition provides an in vivo plasma profile having a AUC_{0-∞} of less than about, e.g., 550 ng·hr/ml, 500 ng·hr/ml, 450 ng·hr/ml, 400 ng·hr/ml, or 350 ng·hr/ml. In embodiments, the second pharmaceutical composition provides an in vivo plasma profile having a AUC_{0-∞} of less than about, e.g., 300 ng·hr/ml, 250 ng·hr/ml, 200 ng·hr/ml, 150 ng·hr/ml, or 100 ng·hr/ml. In embodiments, the first and second pharmaceutical composition are administered wherein the compositions provide improvement of next day functioning of the patient. In embodiments, the first pharmaceutical composition provides improvement in one or more symptom for more than, e.g., 6 hours, 8 hours, 10 hours, 12 hours, 14 hours, 16 hours, 18 hours, 20 hours, 22 hours or 24 hours after administration of the first pharmaceutical composition.

In embodiments, provided herein are methods of treating a developmental disorder and/or a seizure disorder including administering to a patient in need thereof a first pharmaceutical composition including flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding and a second pharmaceutical composition including flupirtine or a pharmaceutically acceptable salt thereof alone or in combination with one or more of retigabine, N-[2-Amino-4-[[(2,4,6-trimethylphenyl)methyl]amino]-phenyl]carbamic acid ethyl ester, gaboxadol, pipradrol, ganaxolone or allopregnanolone, or a pharmaceutically acceptable salt of any of the preceding wherein the first composition provides an in vivo plasma profile with a Cₘₐₓ that is more than about 50% greater than the Cₘₐₓ provided by the administration of the second pharmaceutical composition. As used herein the Cₘₐₓ provided by the administration of the second pharmaceutical composition may or may not include the plasma profile contribution of the first pharmaceutical composition. In embodiments, the administration of the second pharmaceutical composition does not include the plasma profile contribution of the first pharmaceutical composition. In embodiments, the first composition provides an in vivo plasma profile having a Cₘₐₓ that is more than about e.g., 60%, 70%, 80%, or 90% greater than the Cₘₐₓ provided by the administration of the second pharmaceutical composition.

In embodiments, the Tₘₐₓ of the first pharmaceutical composition is less than 3 hours. In embodiments, the Tₘₐₓ of the first pharmaceutical composition is less than 2.5 hours. In embodiments, the Tₘₐₓ of the first pharmaceutical composition is less than 2 hours. In embodiments, the Tₘₐₓ of the first pharmaceutical composition is less than 1.5 hours. In embodiments, the Tₘₐₓ of the first pharmaceutical composition is less than 1 hour. In embodiments, the Tₘₐₓ of the first pharmaceutical composition is less than 0.5 hour. In embodiments, the Tₘₐₓ of the first pharmaceutical composition is less than 0.25 hour. In embodiments, the Tₘₐₓ of the second pharmaceutical composition is less than 3 hours. In embodiments, the Tₘₐₓ of the second pharmaceutical composition is less than 2.5 hours. In embodiments, the Tₘₐₓ of the second pharmaceutical composition is less than 2 hours. In embodiments, the Tₘₐₓ of the second pharmaceutical composition is less than 1.5 hours. In embodiments, the Tₘₐₓ of the second pharmaceutical composition is less than 1 hour. In embodiments, the Tₘₐₓ of the second pharmaceutical composition is less than 0.5 hour. In embodiments, the Tₘₐₓ of the second pharmaceutical composition is less than 0.25 hour.

In embodiments, the first pharmaceutical composition provides a dissolution of at least about 80% within the first 20 minutes of administration to a patient in need thereof. In embodiments, the first pharmaceutical composition provides a dissolution of at least about, e.g., 85%, 90% or 95% within the first 20 minutes of administration to a patient in need thereof. In embodiments, the first pharmaceutical composition provides a dissolution of at least 80% within the first 10 minutes of administration to a patient in need thereof.

In embodiments, administration of the first and second pharmaceutical compositions may be simultaneous or separated by an interval of time to achieve long-term improvement in at least one symptom. In embodiments, the first and second pharmaceutical composition may be administered 1, 2, 3, 4, 5, 6, 7, 8, 9, 10, 11, or 12 hours apart. In embodiments the first and second pharmaceutical composition may be administered 12 hours apart. In embodiments, the first and second pharmaceutical compositions may administered within, e.g., 15 minutes, 30 minutes, 1 hour, 2 hours, 6 hours, 12 hours, 18 hours, 24 hours, etc. In embodiments, the first and second pharmaceutical compositions may administered separated by at least, e.g., 15 minutes, 30 minutes, 1 hour, 2 hours, 12 hours, 18 hours, 24 hours, etc. In embodiments, improvement in at least one symptom of a developmental disorder and/or seizure disorder for more than 8 hours after administration to the patient is provided. In embodiments, improvement for more than about, e.g., 10 hours, 12 hours, 15 hours, 18 hours, 20 hours, 24 hours, 30 hours, 36 hours, 42 hours or 48 hours after administration to the patient is provided.

In embodiments, the administration of the first and second pharmaceutical composition may provide a synergistic effect to improve at least one symptom of a developmental disorder.

In embodiments, the first and/or the second pharmaceutical compositions are administered once, twice, three, four or more times daily, or every other day. In embodiments, the first or the second pharmaceutical composition is provided to the patient in the evening. In embodiments, the second pharmaceutical composition includes an amount of flupirtine or a pharmaceutically acceptable salt thereof alone or in combination with one or more of retigabine, N-[2-Amino-4-[[(2,4,6-trimethylphenyl)methyl]amino]phenyl]carbamic acid ethyl ester, gaboxadol, pipradrol, ganaxolone or allopregnanolone, or a pharmaceutically acceptable salt of any of the preceding that is at least one third of the amount of flupirtine or a pharmaceutically acceptable salt thereof alone or in combination with one or more of retigabine, N-[2-Amino-4-[[(2,4,6-trimethylphenyl)methyl]amino]phenyl]-carbamic acid ethyl ester, gaboxadol, pipradrol, ganaxolone or allopregnanolone, or a pharmaceutically acceptable salt of any of the preceding in the first pharmaceutical composition. In embodiments, the second pharmaceutical composition includes an amount of flupirtine or a pharmaceutically acceptable salt thereof alone or in combination with one or more of retigabine, N-[2-Amino-4-[[(2,4,6-trimethylphenyl)methyl]-amino]phenyl]carbamic acid ethyl ester, gaboxadol, pipradrol, ganaxolone or allopregnanolone, or a pharmaceutically acceptable salt of any of the preceding that is at least half of the amount of flupirtine or a pharmaceutically acceptable salt thereof alone or in combination with one or more of retigabine, N-[2-Amino-4-[[(2,4,6-trimethylphenyl)methyl]amino]phenyl]carbamic acid ethyl ester, gaboxadol, pipradrol, ganaxolone or allopregnanolone, or a pharmaceutically acceptable salt of any of the preceding that is provided in the first pharmaceutical composition.

In embodiments, provided herein are methods of treating a developmental disorder and/or a seizure disorder including administering to a patient in need thereof a first pharmaceutical dosage including a sub-therapeutic amount of flupirtine or a pharmaceutically acceptable salt thereof alone or in combination with a sub-therapeutic amount of one or more of retigabine, N-[2-Amino-4-[[(2,4,6-trimethylphenyl)methyl]amino]phenyl]-carbamic acid ethyl ester, gaboxadol, pipradrol, ganaxolone or allopregnanolone, or a pharmaceutically acceptable salt of any of the preceding. In embodiments, treating a developmental disorder and/or a seizure disorder includes administering to a patient in need thereof a first pharmaceutical dosage including a sub-therapeutic amount of flupirtine or a pharmaceutically acceptable salt thereof alone or in combination with a therapeutic or sub-therapeutic amount of one or more of retigabine, N-[2-Amino-4-[[(2,4,6-trimethylphenyl)-methyl]amino]phenyl]carbamic acid ethyl ester, gaboxadol, pipradrol, ganaxolone or allopregnanolone, or a pharmaceutically acceptable salt of any of the preceding wherein the composition provides improvement in one or more symptoms of the disorder for more than 6 hours after administration.

In embodiments, the first and/or the second pharmaceutical compositions contain sub-therapeutic dosages. A sub-therapeutic dosage is an amount of active substance, e.g., one or more of flupirtine, retigabine, N-[2-Amino-4-[[(2,4,6-trimethylphenyl)-methyl]amino]phenyl]-carbamic acid ethyl ester, gaboxadol, pipradrol, ganaxolone or allopregnanolone, or a pharmaceutically acceptable salt of any of the preceding, that is less than the amount typically required for a therapeutic effect. In embodiments, a sub-therapeutic dosage is an amount of one or more of flupirtine, retigabine, N-[2-Amino-4-[[(2,4,6-trimethylphenyl)-methyl]amino]-phenyl]carbamic acid ethyl ester, gaboxadol, pipradrol, ganaxolone or allopregnanolone, or a pharmaceutically acceptable salt of any of the preceding, that alone may not provide improvement in at least one symptom of the developmental disorder but is sufficient to maintain such improvement. In embodiments, the methods provide administering a first pharmaceutical composition that provides improvement in at least one symptom of a developmental disorder and/or seizure disorder and a second composition that maintains the improvement. In embodiments, the second composition contains a sub-therapeutic dose of one or more of flupirtine, retigabine, N-[2-Amino-4-[[(2,4,6-trimethylphenyl)-methyl]amino]phenyl]-carbamic acid ethyl ester, gaboxadol, pipradrol, ganaxolone or allopregnanolone, or a pharmaceutically acceptable salt of any of the preceding. In embodiments, after administration of the first pharmaceutical composition, the second pharmaceutical composition may provide a synergistic effect to improve at least one symptom of a developmental disorder and/or seizure disorder. In embodiments, the second pharmaceutical composition may provide a synergistic effect to improve at least one symptom of a developmental disorder and/or seizure disorder.

In embodiments, provided herein are methods of treating a developmental disorder and/or seizure disorder including administering to a patient in need thereof a first pharmaceutical composition including a first pharmaceutical dosage of, e.g., flupirtine or a pharmaceutically acceptable salt thereof alone or in combination with one or more of retigabine, N-[2-Amino-4-[[(2,4,6-trimethylphenyl)methyl]amino]phenyl]-carbamic acid ethyl ester, gaboxadol, pipradrol, ganaxolone or allopregnanolone, or a pharmaceutically acceptable salt of any of the preceding, wherein the first pharmaceutical dosage provides improvement for more than 6 hours after administration, and a second pharmaceutical composition including a sub-therapeutic dosage of one or more of flupirtine, retigabine, N-[2-Amino-4-[[(2,4,6-trimethylphenyl)-methyl]amino]phenyl]-carbamic acid ethyl ester, gaboxadol, pipradrol, ganaxolone or allopregnanolone, or a pharmaceutically acceptable salt of any of the preceding.

In embodiments, the first or the second pharmaceutical composition are provided to the patient once in the evening and once in the morning. In embodiments, the total amount of including flupirtine or a pharmaceutically acceptable salt thereof alone or in combination with one or more of retigabine, N-[2-Amino-4-[[(2,4,6-trimethylphenyl)methyl]amino]phenyl]-carbamic acid ethyl ester, gaboxadol, pipradrol, ganaxolone or allopregnanolone, or a pharmaceutically acceptable salt of any of the preceding administered to a subject in a 24-hour period is any of the respective amounts described herein.

In embodiments, the first and/or the second pharmaceutical compositions may be provided with conventional release or modified release profiles. The first and second pharmaceutical compositions may be provided at the same time or separated by an interval of time, e.g., 1 hour, 2 hours, 3 hours, 4 hours, 5 hours, 6 hours, 12 hours, etc. In embodiments, the first and the second pharmaceutical compositions may be provided with different drug release profiles to create a two-phase release profile. For example, the first pharmaceutical composition may be provided with an immediate release profile, e.g., ODDF, parenteral, etc., and the second pharmaceutical composition may provide an extended release profile. In embodiments, one or both of the first and second pharmaceutical compositions may be provided with an extended release or delayed release profile. Such compositions may be provided as pulsatile formulations, multilayer tablets or capsules containing tablets, beads, granules, etc. In embodiments, the first pharmaceutical composition is an immediate release composition. In embodiments, the second pharmaceutical composition is an immediate release composition. In embodiments, the first and second pharmaceutical compositions are provided as separate immediate release compositions, e.g., film, tablets or capsules. In embodiments the first and second pharmaceutical compositions are provided 12 hours apart.

It should be understood that respective dosage amounts of flupirtine, retigabine, N-[2-Amino-4-[[(2,4,6-trimethylphenyl)-methyl]amino]phenyl]-carbamic acid ethyl ester, gaboxadol, pipradrol, ganaxolone or allopregnanolone, or a pharmaceutically acceptable salt of any of the preceding, that are provided herein are applicable to all the dosage forms described herein including conventional dosage forms, modified dosage forms, the first and second pharmaceutical compositions, as well as the parenteral formulations described herein. Those skilled in the art will determine appropriate amounts depending on criteria such as dosage form, route of administration, patient tolerance, efficacy, therapeutic goal and therapeutic benefit, among other pharmaceutically acceptable criteria.

Combination therapies utilizing flupirtine, and one or more of retigabine, N-[2-Amino-4-[[(2,4,6-trimethylphenyl)-methyl]amino]phenyl]-carbamic acid ethyl ester, gaboxadol, pipradrol, ganaxolone or allopregnanolone, or a pharmaceutically acceptable salt of any of the preceding, can include administration of the active agents together in the same admixture, or in separate admixtures. In embodiments, the pharmaceutical composition can includes two, three, or more active agents. In embodiments, the combinations result in a more than additive effect on the treatment of the disease or disorder. Thus, treatment is provided of a developmental disorder and/or seizure disorder with a combination of agents that combined, may provide a synergistic effect that enhances efficacy.

In embodiments, a co-therapy of flupirtine, and one or more of retigabine, N-[2-Amino-4-[[(2,4,6-trimethylphenyl)-methyl]amino]phenyl]-carbamic acid ethyl ester, gaboxadol, pipradrol, ganaxolone or allopregnanolone, or a pharmaceutically acceptable salt of any of the preceding, is effective to reduce frequency or severity of symptoms in the subject greater than any of the compounds administered alone. In embodiments, the co-therapy produces a more than additive result compared to compounds administered individually.

In embodiments, the subject may be started at a low dose and the dosage is escalated. In this manner, it can be determined if the drug is well tolerated in the subject. Dosages can be lower for children than for adults.

In embodiments, such as combination therapies, a dose of flupirtine, retigabine, N-[2-Amino-4-[[(2,4,6-trimethylphenyl)-methyl]amino]phenyl]-carbamic acid ethyl ester, gaboxadol, pipradrol, ganaxolone or allopregnanolone, or a pharmaceutically acceptable salt of any of the preceding, for children can be, e.g., 0.01 mg/kg to 0.1 mg/kg or 0.1 mg/kg to 1 mg/kg. For example, the weight/weight ratio of flupirtine to gaboxadol or flupirtine to N-[2-Amino-4-[[(2,4,6-trimethylphenyl)methyl]amino]phenyl]-carbamic acid-ethyl ester or a pharmaceutically acceptable salt thereof can be 10-to-1. In embodiments, the dosing ratio based on milligrams of active pharmaceutical ingredient (API) can range from 0.1-to-1 to 100-to-1 of flupirtine to gaboxadol or to N-[2-Amino-4-[[(2,4,6-trimethylphenyl)methyl]amino]phenyl]-carbamic acid-ethyl ester or a pharmaceutically acceptable salt thereof, respectively.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value. Alternatively, particularly with respect to biological systems or processes, the term can mean within an order of magnitude, preferably within 5-fold, and more preferably within 2-fold, of a value.

“Improvement” refers to the treatment of a developmental disorder such as Autistic Spectrum Disorder, pervasive developmental disorder, autism, Angelman syndrome, Fragile X syndrome, Fragile X-associated tremor/ataxia syndrome (FXTAS), Rett syndrome, Asperger's syndrome, Childhood Disintegrative Disorder, Attention-deficit/hyperactivity disorder (ADHD), Prader-Willi Syndrome, Landau-Kleffner Syndrome, Rasmussen's syndrome, Dravet syndrome, Tardive Dyskinesia, Williams Syndrome and/or seizure disorders per se, and/or seizure disorders independent of, or associated with, any of the foregoing developmental disorders, and/or seizure disorders such as epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, increased seizure activity or breakthrough seizures (also called serial or cluster seizures), measured relative to at least one symptom of the foregoing disorders.

“Improvement in next day functioning” or “wherein there is improvement in next day functioning” refers to improvement after waking from an overnight sleep period wherein the beneficial effect of administration of flupirtine or a pharmaceutically acceptable salt thereof alone or in combination with retigabine, N-[2-Amino-4-[[(2,4,6-trimethylphenyl)methyl]amino]phenyl]-carbamic acid ethyl ester, gaboxadol, pipradrol, ganaxolone or allopregnanolone, or a pharmaceutically acceptable salt of any of the preceding, applies to at least one symptom of a disorder herein and is discernable, either subjectively by a patient or objectively by an observer, for a period of time, e.g., 2 hours, 3 hours, 4 hours, 5 hours, 6 hours, 12 hours, 24 hours, etc. after waking.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng□hr/ml or μg·hr/ml). The area under the curve is governed by clearance. Clearance is defined as the volume of blood or plasma that is totally cleared of its content of drug per unit time (ml/min).

“Treating” or “treatment” refers to alleviating or delaying the appearance of clinical symptoms of a disease or condition in a subject that may be afflicted with or predisposed to the disease or condition, but does not yet experience or display clinical or subclinical symptoms of the disease or condition. In certain embodiments, “treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” also refers to inhibiting the disease or condition, e.g., arresting or reducing its development or at least one clinical or subclinical symptom thereof. “Treating” or “treatment” further refers to relieving the disease or condition, e.g., causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic (curative) treatment are two separate embodiments of the disclosure herein.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount” means a dosage sufficient to alleviate one or more symptoms of a disorder, disease, or condition being treated, or to otherwise provide a desired pharmacological and/or physiologic effect. “Effective amount” or “therapeutically effective amount” may be used interchangeably herein.

“Co-administered with”, “administered in combination with”, “a combination of” or “administered along with” may be used interchangeably and mean that two or more agents are administered in the course of therapy. The agents may be administered together at the same time or separately in spaced apart intervals. The agents may be administered in a single dosage form or in separate dosage forms.

“Patient in need thereof” may include individuals that have been diagnosed with a developmental disorder such as Autistic Spectrum Disorder, pervasive developmental disorder, autism, Angelman syndrome, Fragile X syndrome, Fragile X-associated tremor/ataxia syndrome (FXTAS), Rett syndrome, Asperger's syndrome, Childhood Disintegrative Disorder, Attention-deficit/hyperactivity disorder (ADHD), Prader-Willi Syndrome, Landau-Kleffner Syndrome, Rasmussen's syndrome, Dravet syndrome, Tardive Dyskinesia, Williams Syndrome and/or seizure disorders per se, and/or seizure disorders independent of, or associated with, any of the foregoing developmental disorders, and/or seizure disorders such as epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, increased seizure activity or breakthrough seizures (also called serial or cluster seizures). The methods may be provided to any individual including, e.g., wherein the patient is a neonate, infant, a pediatric patient (6 months to 12 years), an adolescent patient (age 12-18 years) or an adult (over 18 years).

“Prodrug” refers to a pharmacological substance (drug) that is administered to a subject in an inactive (or significantly less active) form. Once administered, the prodrug is metabolized in the body (in vivo) into a compound having the desired pharmacological activity.

“Analog” and “Derivative” may be used interchangeably and refer to a compound that possesses the same core as the parent compound, but may differ from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

The term “pharmaceutically acceptable salt”, as used herein, refers to derivatives of the compounds defined herein, wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts. The pharmaceutically acceptable salts can be synthesized from the parent compound, which contains a basic or acidic moiety, by conventional chemical methods.

### EXAMPLES

The examples provided herein are included solely for augmenting the disclosure herein and should not be considered to be limiting in any respect.

### Example 1

### Prospective Assessment of the Efficacy of Flupirtine in Patients with Angelman Syndrome

This study is designed to determine whether flupirtine or a pharmaceutically acceptable salt thereof will lead to an improvement in one or more symptoms of Angelman syndrome. Participants are randomized into 6 separate treatment groups (A-F). Inclusion criteria for randomization will require that each participant has been previously diagnosed with Angelman syndrome by clinical evaluation or that the participant is diagnosed with one or more of the major and minor criteria for Angelman syndrome.

Major Criteria include:
Functionally severe developmental delay
Speech impairment; none or minimal words used
Movement or balance disorder
Behavioral uniqueness, frequent laughs/smiling, excitable personality, hand flapping, short attention span

Minor Criteria include:
Deceleration in head circumference growth (post-natal)
Seizures (myoclonic, absence, drop, tonic-clonic)
Abnormal EEG (with patterns suggestive of AS, or hypsarrhythmia)
Sleep disturbance
Attraction to or fascination with water
Drooling

After randomization the participants are placed into 6 separate treatment groups (A-F) and a placebo group. Treatment group A receives 100 mg flupirtine orally in the morning for two weeks. Treatment group B receives 100 mg flupirtine intravenously in the morning for two weeks. Treatment group C receives 400 mg flupirtine orally in the evening for two weeks. Treatment group D receives 100 mg flupirtine orally in the morning and 100 mg flupirtine orally in the evening for two weeks. Treatment group E receives 100 mg flupirtine intravenously in the morning and 100 mg flupirtine intravenously in the evening for two weeks. Treatment group F receives 400 mg flupirtine orally in the morning and 400 mg flupirtine in the evening for two weeks.

Participants are assessed throughout the treatment period to determine whether flupirtine administration leads to an improvement in one or more symptoms of Angelman syndrome. Several behavioral domains; communication, attention, maladaptive behaviors, and hyper-excitability are assessed. To quantify the communication behavior, participants engage in an unstructured play session to elicit speech and non-verbal communication attempts. Speech attempts by the child are transcribed phonetically and categorized into five different types of vocalizations using the Stark Assessment of Early Vocal Development-Revised (SAEVD-R) (Nathani, Ertmer et al. 2006) which categorizes non-speech and pre-speech sounds (protophones), as well as vowels, consonants and syllables.

Gait abnormalities occur in most cases of Angelman syndrome. Thus, five primary spatiotemporal parameters are analyzed: cadence, gait velocity, stride width, step length and percent stance. For each parameter, a principal component analysis is used to establish a gait index for assessment of the subjects.

In addition, primary outcome measures that may be assessed include changes in raw or standard scores between baseline and after trial completion of:
I. Bayley Scales of Infant and Toddler Development, 3rd edition (or the Mullen Scales of Early Learning in the more developmentally advanced subjects);
II. Vineland Adaptive Behavior Scales, 2nd edition (standard scores only);
III. Preschool Language Scale, 4th edition;
IV. Aberrant Behavior Checklist—Community version; and
V. A change from baseline in the Clinical Global Impressions Severity Scale Score.

Secondary outcome measures may include normalization of the electroencephalogram (EEG) signature when comparing post flupirtine administration results to baseline results.

### Example 2

### Prospective Assessment of the Efficacy of Retigabine in Pediatric Patients with Status Epilepticus

This study is designed to determine whether retigabine leads to an improvement in one or more symptoms of status epilepticus. Epilepsy is among the most common serious neurologic disorders in childhood. Medicines with novel actions of mechanisms of action are needed to try to address the unmet clinical need for seizure control in patients with status epilepticus. In addition, the purpose of this study is to evaluate the safety and tolerability of retigabine as treatment in subjects with status epilepticus.

Retigabine or a pharmaceutically acceptable salt thereof will be administered intravenously to pediatric subjects (3 months to 16 years) in amounts ranging from 0.01, 0.05, 0.1, 0.25, 0.5, 0.75, 1, 2.5, 5, 7.5, 10, 25, and 50 mg as 50 mL short-term infusion solution intravenously (IV) within 15 minutes (infusion rate 200 mL/h). Subjects whose seizure does not stop or recurs within 10 minutes after the initial dose may receive the same amount of retigabine injection no earlier than 10 minutes following the initial dose. Subjects whose seizure stops within 10 minutes after the initial dose, but recurs thereafter (within 12 hours) may receive the same amount of retigabine injection; a total of 2 doses will be permitted in this study.

### Inclusion Criteria:

Subjects with status epilepticus or repetitive status epilepticus/cluster seizure who have seizures that can be evaluated by investigator's visual observations based on motor symptoms or who have seizures that can be evaluated by EEG.
Subjects with status epilepticus accompanied by generalized seizure, partial seizure or secondarily generalized seizure lasting 5 minutes or longer
Subjects with repetitive status epilepticus/cluster seizure accompanied by not less than 3 consecutive episodes of generalized seizure, partial seizure or secondarily generalized seizure in 1 hour.
Subjects not younger than 3 months (either gender is eligible for the study)

### Primary Outcome Measures:

Percentage of Participants With Clinical Benefit [Time Frame: 30 minutes]
Percent of participants whose initial seizure stopped within 10 minutes after initial dose and who continued seizure-free for at least 30 minutes after the completion of initial dose (responder rate)

### Secondary Outcome Measures:

Percent of participants whose initial seizure stopped within 10 minutes after the administration of retigabine (either initial or second dose [in 10 to 30 minutes from the initial dose]) and who continued seizure-free for at least 30 minutes. [Time Frame: 1 hour]
Percent of participants whose seizures stopped within 10 minutes after the administration of retigabine (only the initial dose) and who continued seizure-free for at least 12 hours post-dose. [Time Frame: 12 hours]
Percent of participants whose seizures stopped within 10 minutes after the administration of retigabine (either initial or second dose [in 10 to 30 minutes from the initial dose]) and who continued seizure-free for at least 12 hours post-dose. [Time Frame: 12 hours]
Percent of participants whose seizures stopped within 10 minutes after the administration of retigabine (only the initial dose) and who continued seizure-free for at least 24 hours post-dose. [Time Frame: 24 hours]
Percent of participants whose seizures stopped within 10 minutes after the administration of retigabine (either initial or second dose [in 10 to 30 minutes from the initial dose]) and who continued seizure-free for at least 24 hours post-dose. [Time Frame: 24 hours]
Time to resolution of seizures from the administration of retigabine (only the initial dose). [Time Frame: 24 hours]
Time to resolution of seizures from the administration of retigabine (either initial or second dose). [Time Frame: 24 hours]
Time to relapse from the resolution of seizures following the administration of retigabine (only the initial dose, within 24 hours). [Time Frame: 24 hours]
Time to relapse from the resolution of seizures following the administration of retigabine (either initial or second dose, within 24 hours). [Time Frame: 24 hours]

### Example 3

### Prospective Assessment of the Efficacy of Flupirtine in Pediatric Patients with Status Epilepticus

This study is designed to determine whether flupirtine leads to an improvement in one or more symptoms of status epilepticus. Epilepsy is among the most common serious neurologic disorders in childhood. Medicines with novel actions of mechanisms of action are needed to try to address the unmet clinical need for seizure control in patients with status epilepticus. In addition, the purpose of this study is to evaluate the safety and tolerability of flupirtine as treatment in subjects with status epilepticus.

Flupirtine or a pharmaceutically acceptable salt thereof such as maleate or tartrate will be administered intravenously to pediatric subjects (3 months to 16 years) in amounts ranging from 1, 2.5, 5, 7.5, 10, 25, 50, 75 and 100 mg as 50 mL short-term infusion solution intravenously (IV) within 15 minutes (infusion rate 200 mL/h). Subjects whose seizure does not stop or recurs within 10 minutes after the initial dose may receive the same amount of flupirtine injection no earlier than 10 minutes following the initial dose. Subjects whose seizure stops within 10 minutes after the initial dose, but recurs thereafter (within 12 hours) may receive the same amount of flupirtine injection; a total of 2 doses will be permitted in this study.

### Inclusion Criteria:

Subjects with status epilepticus or repetitive status epilepticus/cluster seizure who have seizures that can be evaluated by investigator's visual observations based on motor symptoms or who have seizures that can be evaluated by EEG.
Subjects with status epilepticus accompanied by generalized seizure, partial seizure or secondarily generalized seizure lasting 5 minutes or longer
Subjects with repetitive status epilepticus/cluster seizure accompanied by not less than 3 consecutive episodes of generalized seizure, partial seizure or secondarily generalized seizure in 1 hour.
Subjects not younger than 3 months (either gender is eligible for the study)

### Primary Outcome Measures:

Percentage of Participants With Clinical Benefit [Time Frame: 30 minutes]
Percent of participants whose initial seizure stopped within 10 minutes after initial dose and who continued seizure-free for at least 30 minutes after the completion of initial dose (responder rate)

### Secondary Outcome Measures:

Percent of participants whose initial seizure stopped within 10 minutes after the administration of flupirtine (either initial or second dose [in 10 to 30 minutes from the initial dose]) and who continued seizure-free for at least 30 minutes. [Time Frame: 1 hour]
Percent of participants whose seizures stopped within 10 minutes after the administration of flupirtine (only the initial dose) and who continued seizure-free for at least 12 hours post-dose. [Time Frame: 12 hours]
Percent of participants whose seizures stopped within 10 minutes after the administration of flupirtine (either initial or second dose [in 10 to 30 minutes from the initial dose]) and who continued seizure-free for at least 12 hours post-dose. [Time Frame: 12 hours]
Percent of participants whose seizures stopped within 10 minutes after the administration of flupirtine (only the initial dose) and who continued seizure-free for at least 24 hours post-dose. [Time Frame: 24 hours]
Percent of participants whose seizures stopped within 10 minutes after the administration of flupirtine (either initial or second dose [in 10 to 30 minutes from the initial dose]) and who continued seizure-free for at least 24 hours post-dose. [Time Frame: 24 hours]
Time to resolution of seizures from the administration of flupirtine (only the initial dose). [Time Frame: 24 hours]
Time to resolution of seizures from the administration of flupirtine (either initial or second dose). [Time Frame: 24 hours]
Time to relapse from the resolution of seizures following the administration of flupirtine (only the initial dose, within 24 hours). [Time Frame: 24 hours]
Time to relapse from the resolution of seizures following the administration of flupirtine (either initial or second dose, within 24 hours). [Time Frame: 24 hours]

### Example 4

### Plasma Concentration Profiles and Dose Proportionality of Gaboxadol Monohydrate

The following Example provides the plasma concentration profiles and dose proportionality of gaboxadol monohydrate following single oral doses ranging from 2.5 to 20 mg. The absolute bioavailability of gaboxadol monohydrate capsules ranging from 2.5 to 20 mg is also assessed.

This study was composed of separate groups of 10 healthy adult subjects (at least 4 of each gender) who participated in a 6-period, double-blind, randomized, crossover study designed to access the dose proportionality and absolute bioavailability of 5 single oral doses of gaboxadol across the dose range of 2.5 to 20 mg. The order in which the subjects received the 5 single oral doses of gaboxadol (2.5; 5; 10; 15; and 20 mg) was randomized within Treatment Periods 1 through 5. Each subject was expected to complete all 6 treatment periods and there was a washout of at least 4 days between each treatment period.

Each oral dosing within Treatment Periods consisted of 2 capsules of test drug taken simultaneously at each scheduled dosing. The treatment designations for the orally administered study drugs were as follows: Treatment A—one 2.5 mg gaboxadol capsule and 1 matching placebo capsule; Treatment B—one 5 mg gaboxadol capsule and 1 matching placebo capsule; Treatment C—one 10 mg gaboxadol capsule and 1 matching placebo capsule; Treatment D—one 15 mg gaboxadol capsule and 1 matching placebo capsule; and Treatment E—20 mg gaboxadol (two 10 mg gaboxadol capsules). Subjects received their study drug after an overnight fast with 240 mL of water in the morning about 8:00 AM. Water was permitted ad libitum except within 1 hour prior to and after study drug administration. No food was allowed for 4 hours post dose.

For each subject in each treatment, plasma and urine samples were collected over 16 hours post-dosing for the determination of pharmacokinetic parameters (e.g., AUC, Cₘₐₓ, Tₘₐₓ, apparent t_{1/2}, cumulative urinary excretion, renal clearance, clearance, and steady-state volume of distribution, as appropriate). AUC and Cₘₐₓ for gaboxadol were potency adjusted to facilitate comparison of pharmacokinetic data across studies. Table 1 provides the individual potency-adjusted pharmacokinetic parameters of gaboxadol following single oral doses (2.5, 5, 10, 15, and 20 mg).

TABLE 1
Pharmacokinetic parameters for gaboxadol following oral and IV administration

| | Geometric Mean (N = 10) | | | | | | |
|---|---|---|---|---|---|---|---|
| | | | 10 mg | 10 mg | | | |
| Parameter | 2.5 mg | 5 mg | Oral | I.V. | 15 mg | 20 mg | Slope (90% CI)^{††} |
| AUC_{0-∞} (ng · hr/mL) | 90 | 171 | 346 | 380 | 539 | 669 | 0.98 (0.95, 1.01) |
| Cₘₐₓ (ng/mL)^{†} | 61 | 110 | 232 | 212 | 382 | 393 | 0.95 (0.88, 1.02) |
| Tₘₐₓ (hr)^{‡} | 0.5 | 0.6 | 0.5 | — | 0.5 | 0.6 | |
| Apparent t_{1/2} (hr)^{§} | 1.5 | 1.5 | 1.6 | 1.5 | 1.5 | 1.6 | |
| CL/F (mL/min)^{¶} | 461 | 488 | 476 | 438 | 469 | 499 | |
| fₑ(%) | 43 | 45 | 53 | 53 | 50 | 53 | |
| CL_{R}(mL/min) | 196 | 222 | 250 | 208 | 234 | 265 | |

| | |
|---|---|
| F (%) (90% CI)^{#} | 92% (0.86, 0.97) |

^{†}Cₘₐₓ (ng/mL) for 10 mg IV.
^{‡}Median.
^{§}Harmonic Mean.
^{¶}CL (mL/min) for 10 mg IV.
^{#}Bioavailability relative to 10 mg I.V. reference based on pooled dose-adjusted (to 10 mg) oral AUC_{0-∞} values.
^{††}Dose proportionality assessment of oral treatments only.

The bioavailability of gaboxadol is approximately 92%. Plasma AUC_{0-∞} and Cₘₐₓ of gaboxadol show dose proportional increases and appear to be linear over the entire dose range examined, from of 2.5 to 20 mg. The time to peak plasma concentrations (Tₘₐₓ 30-60 min) and the half-life (t½ of 1.5 h) for gaboxadol appear to be independent of dose across the gaboxadol dose range of 2.5 to 20 mg. The excretion of gaboxadol is mainly via urine, where 96.5% of the dose is recovered; 75% is recovered within 4 hours after administration.

### Example 5

### Prospective Assessment of the Efficacy of Pipradrol in Patients with Fragile X Syndrome

This study is designed to determine whether pipradrol leads to an improvement in one or more symptoms of Fragile X syndrome. Participants are randomized into 6 separate treatment groups (A-F). Inclusion criteria for randomization require patients that have been diagnosed with Fragile X syndrome. For example, patients who are at least moderately ill based on a Clinical Global Impression Severity score of at least 4 and have qualifying scores on the ABC-C and IQ test

After randomization the participants are placed into 7 separate treatment groups (A-F) and a placebo group. Treatment group A receives 0.5 mg pipradrol in the morning. Treatment group B receives 1 mg pipradrol in the morning. Treatment group C receives 2 mg pipradrol in the morning. Treatment group D receives 3 mg pipradrol in the morning. Treatment group E receives 4 mg pipradrol in the morning. Treatment group F receives 2 mg pipradrol in the morning and 2 mg pipradrol in the afternoon. Treatment group F receives 3 mg in the morning and 2 mg in the afternoon.

Participants are assessed throughout the treatment period to determine whether administration pipradrol leads to an improvement in one or more symptoms of Fragile X syndrome. In particular, patients are assessed using one or more primary and secondary outcome measures. Primary Outcome Measures may include:

Change From Baseline in Behavioral Symptoms of Fragile X Syndrome Using the Aberrant Behavior Checklist-Community Edition (ABC-CFX) Total Score;

Global Improvement of Symptoms in Fragile X Using the Clinical Global Impression-Improvement (CGI-I) Scale;

Change From Baseline in Irritability, Lethargy/Withdrawal, Stereotypic Behavior, Hyperactivity, Inappropriate Speech and Social Avoidance Assessed by the Individual Subscales of the ABC-CFX Scale;

Change From Baseline in Repetitive Behaviors Assessed Using the Repetitive Behavior Scale-Revised (RBS-R) Scores;

Visual Analogue Scale (Behavior); Expressive Vocabulary Test; Vineland Adaptive Behavior Scale-II (VABS-II) Adaptive Behavior Composite Score; and Aberrant Behavior Checklist-Community Edition (ABC-C) Composite Score.

### Example 6

### Prospective Assessment of the Efficacy of N-[2-Amino-4-[[(2,4,6-trimethylphenyl)methyl]amino]phenyl]-carbamic Acid-Ethyl Ester in Patients with Fragile X Syndrome

This study is designed to determine whether N-[2-Amino-4-[[(2,4,6-trimethylphenyl)methyl]amino]phenyl]-carbamic acid-ethyl ester leads to an improvement in one or more symptoms of Fragile X syndrome. Participants are randomized into 6 separate treatment groups (A-F). Inclusion criteria for randomization require patients that have been diagnosed with Fragile X syndrome. For example, patients who are at least moderately ill based on a Clinical Global Impression Severity score of at least 4 and have qualifying scores on the ABC-C and IQ test

After randomization the participants are placed into 6 separate treatment groups (A-F) and a placebo group. Treatment group A receives 5 mg N-[2-Amino-4-[[(2,4,6-trimethylphenyl)methyl]amino]phenyl]-carbamic acid-ethyl ester in the morning. Treatment group B receives 10 mg N-[2-Amino-4-[[(2,4,6-trimethylphenyl)methyl]amino]phenyl]-carbamic acid-ethyl ester in the morning. Treatment group C receives 5 mg N-[2-Amino-4-[[(2,4,6-trimethylphenyl)methyl]amino]phenyl]-carbamic acid-ethyl ester in the morning and 10 mg N-[2-Amino-4-[[(2,4,6-trimethylphenyl)methyl]amino]phenyl]-carbamic acid-ethyl ester in the evening. Treatment group D receives 20 mg N-[2-Amino-4-[[(2,4,6-trimethylphenyl)methyl]amino]phenyl]-carbamic acid-ethyl ester in the morning. Treatment group E receives 10 mg N-[2-Amino-4-[[(2,4,6-trimethylphenyl)methyl]amino]phenyl]-carbamic acid-ethyl ester in the morning and 10 mg N-[2-Amino-4-[[(2,4,6-trimethylphenyl)methyl]-amino]phenyl]-carbamic acid-ethyl ester in the morning. Treatment group F receives 20 mg N-[2-Amino-4-[[(2,4,6-trimethylphenyl)methyl]amino]phenyl]-carbamic acid-ethyl ester in the morning and 20 mg N-[2-Amino-4-[[(2,4,6-trimethylphenyl)methyl]amino]phenyl]-carbamic acid-ethyl ester in the evening.

Participants are assessed throughout the treatment period to determine whether administration of N-[2-Amino-4-[[(2,4,6-trimethylphenyl)methyl]-amino]phenyl]carbamic acid-ethyl ester or a pharmaceutically acceptable salt thereof leads to an improvement in one or more symptoms of Fragile X syndrome. In particular, patients are assessed using one or more primary and secondary outcome measures. Primary Outcome Measures may include:

Change From Baseline in Behavioral Symptoms of Fragile X Syndrome Using the Aberrant Behavior Checklist-Community Edition (ABC-CFX) Total Score;

Global Improvement of Symptoms in Fragile X Using the Clinical Global Impression-Improvement (CGI-I) Scale;

Change From Baseline in Irritability, Lethargy/Withdrawal, Stereotypic Behavior, Hyperactivity, Inappropriate Speech and Social Avoidance Assessed by the Individual Subscales of the ABC-CFX Scale;

Change From Baseline in Repetitive Behaviors Assessed Using the Repetitive Behavior Scale-Revised (RBS-R) Scores;

Visual Analogue Scale (Behavior); Expressive Vocabulary Test; Vineland Adaptive Behavior Scale-II (VABS-II) Adaptive Behavior Composite Score; and Aberrant Behavior Checklist-Community Edition (ABC-C) Composite Score.

### Example 7

### Prospective Assessment of the Efficacy of Ganaxolone in Patients with Fragile X-Associated Tremor/Ataxia Syndrome

This protocol is directed to treating symptomatic permutation carriers who have pre-FXTAS or FXTAS symptoms including neuropathy, central pain symptoms, insomnia, and full FXTAS involving tremor and ataxia which is often associated with cognitive decline.

This will be a two-site study. Participants will be individuals with the premutation and FXTAS. FMR1 CGG repeat lengths will be quantified in all subjects using conventional procedures. FXTAS will be diagnosed following published criteria (Bacalman et al., Clin Psychiatry 2006, 67:87-94; Jacquemont et al., Lancet Neurol 2003, 6:45-55). The study will involve a controlled trial of ganaxolone lasting three months followed by a three month open-label so that those individuals that were treated for the first three months ganaxolone would continue for a second three months and those individuals on placebo would go on ganaxolone for the second three months. Each site would enroll 20 patients per year for a total of 40 at each site over a two year period and between the sites there would be 80 patients participating.

Identical appearing tablets containing either ganaxolone or placebo will be administered. After randomization the participants are randomized into 6 separate treatment groups and a placebo group. Treatment group A receives 100 mg ganaxolone twice daily for the first 3 days, 200 mg ganaxolone twice daily for the next 3 days and 400 mg ganaxolone twice daily for the remainder of the study. Treatment group B receives 150 mg ganaxolone twice daily for the first 3 days, 300 mg ganaxolone twice daily for the next 3 days and 500 mg ganaxolone twice daily for the remainder of the study. Treatment group C receives 200 mg ganaxolone twice daily for the first 3 days, 400 mg ganaxolone twice daily for the next 3 days and 600 mg ganaxolone twice daily for the remainder of the study. Treatment group D receives 200 mg ganaxolone twice daily for the first 3 days, 400 mg ganaxolone twice daily for the next 3 days and 600 mg ganaxolone twice daily for the remainder of the study. Treatment group E receives 200 mg ganaxolone in the morning and 400 mg ganaxolone in the evening 3 months. Treatment group F receives 200 mg of ganaxolone 3 times daily for the first 3 days, 300 mg ganaxolone 3 times daily for the next 3 days and 500 mg 3 times daily for the remainder of the study.

At baseline, and then at three months, and then at six months, the following studies would be done: An assessment of the severity of pain using a pain index and documentation of the type of pain; and a sleep diary will be implemented. Quantitative measures will be implemented using an actometer to observe the severity of sleep disturbances over a one week period of time. Neuropsychological measures would include the Mini-Mental State Examination (MMSE), Behavioral Dyscontrol Scale (BDS-II), Wechsler Memory Scale IV, the California Verbal Learning Test 2 (CVLT-2), Repeatable Battery for the Assessment of Neuropsychological Status (RBANS) and the SCL-90 for a determination of emotional improvements. Any improvements in the MMSE, the BDS-II, and in event related potential (ERP) studies, particularly with the N4 Repetition Paradigm, and in volumetric changes in the hippocampus will be assessed. Motor assessments will be made which documents abnormalities in those with FXTAS compared to other movement disorders. An FXTAS rating scale will be utilized. MM volumetric studies with the 3 Tesla MRI along with DTIs will be conducted. Eye-tracking measures looking at an inhibitory paradigm will be evaluated. The P6 repetition effect over a six month will be evaluated. All of these measures will be at baseline, three months, and six months. Baseline cognitive testing using the Wechsler Scale and WAIS-IV will be carried out also. This could be repeated after one year but typically not sooner. Improvement in neuropathy may be detected and followed through clinical examination using neurodiagnostic studies or electrophysiological studies.

### Example 8

### Prospective Assessment of the Efficacy of Allopregnanolone in Patients with Fragile X-Associated Tremor/Ataxia Syndrome

This study is designed to determine whether allopregnanolone leads to an improvement in cognitive symptoms, i.e., attentional processes which are fundamental to executive function/dysfunction associated with Fragile X-associated tremor/ataxia syndrome (FXTAS) and involves a placebo-controlled, double-blind, randomized clinical trial and an auditory “oddball” task. Participants will be individuals with FXTAS. FMR1 CGG repeat lengths will be quantified in all subjects using conventional procedures. FXTAS will be diagnosed following published criteria (Bacalman et al., Clin Psychiatry 2006, 67:87-94; Jacquemont et al., Lancet Neurol 2003, 6:45-55). For the main allopregnanolone trial, 200 potential participants will be screened for eligibility. Randomization to either placebo or allopregnanolone will be blinded to all study personnel, investigators, and participants until the end of the one year trial period. Participants will participate in an auditory “oddball”/event related potentials (ERPs) experiment.

Identical appearing intravenous injections containing either allopregnanolone or placebo will be administered. After randomization the participants are randomized into 3 separate treatment groups (A-C) and a placebo group. Treatment group A will receive 2 mg allopregnanolone once a week for 3 weeks. Treatment group B receives 2 mg allopregnanolone weekly for the first two weeks and 4 mg the third week. Treatment group C will receive and intravenous infusion of allopregnanolone at escalating doses of 2 mg, 4 mg, and 6 mg once weekly over a three week period. The highest dose tolerated without sedation will be held stable for the remaining weekly infusions, for a total of 12 infusions.

In the auditory “oddball” experiment, patients will be instructed to detect an infrequent “oddball” tone embedded in a train of non-target standard tones. Subjects will press a button to each target detected and also keep a mental count of the number of targets in that experimental block. Prior studies in premutation carriers using the same “oddball” paradigm have demonstrated an altered frontal P300 (P3) ERP component in FXTAS patients, which tracks their executive dysfunction. See, Yang et al., Ann Neurol 74, 275-283 (2013); Yang et al., Cereb Cortex 23, 2657-2666 (2013). In these studies and others, the earlier abnormalities of prolonged N100 latency and reduced P200 (P2) amplitude were also found in a predominately male FXTAS group but not in female premutation carriers asymptomatic of FXTAS9.

Neuropsychological testing will involve examining each patient's EEG. Accordingly, EEG during a two-stimulus auditory oddball experiment will be recorded in a sound-attenuated, dimly-lit chamber. Lower (113 Hz) and higher (200 Hz) frequency pure tones will be presented at 40 dB above individual hearing level in 4 blocks, each containing 100 tones, with a stimulus onset asynchrony jittered from 1.0-1.5 seconds. Prior to each block, subjects will be instructed to respond to the infrequent (probability equaling 25%) “oddball” tones (high or low target tones, counterbalanced across blocks). A dual task will be employed in which subjects are instructed to press a button to each target tone, and to also keep a mental count of the number of targets in each block. The mental count of target tones will be reported immediately following completion after each block. 32-channel EEG will be recorded with a Nicolet-SM-2000 amplifier (band-pass=0.016-100 Hz, sampled at 250 Hz). Data Analysis will involve the |count-hit| discrepancy in each block (i.e., the absolute value of the difference between correct button-presses and mental count to target tones within a block) will be calculated for each participant, as an inverse measure (i.e., a lower value represents better performance) of attention/working memory performance during the oddball task. Event-locked EEG segments contaminated with blinks, eye movements, excessive muscle activity, or amplifier blocking will be rejected using a semi-automated computer algorithm. Artifact-free EEG segments of 1024 ms (with a 100 ms pre-stimulus baseline period, and 924 ms post-stimulus onset) will be averaged by experimental condition to obtain the ERPs. Mean amplitude and local peak latency of 4 ERP components will be quantified in the following time windows: N100 (N1, 70-150 ms), P2 (160-260 ms), N200 (N2, 170-300 ms), and P3 (300-650 ms). The waveforms to both target and standard tones will be used to measure N1. The P2 will be measured from ERPs to standard tones. The N2 component is defined from the difference wave (ERPs to targets minus standards). The P3 will be measured from both the difference wave and the ERP waveform to targets. ERP measures will be submitted to repeated-measures ANOVAs (SPSS 22, IBM) with the between-subjects factor of treatment, and the within-subjects factors of visit and electrode. Analyses of N1 and P2 will include 4 fronto-central electrodes (Fz, Cz, FC1/2). Five central channels (Cz, FC1/2, CP1/2) will be used for the N2 analyses. P3 analyses will be carried out with 26 scalp electrodes (all except FP1/2). The Greenhouse-Geiser correction will be used to adjust for violations of sphericity, where appropriate. To further characterize the modulatory effects of allopregnanolone on the P2 component, a habituation analysis will be conducted for P2 amplitude. P2 mean amplitude in response to the first 30 standard tones will be compared to the amplitude of response to the last 30 standard tones within the first block of each study, with the between-subjects factor of treatment, and the within-subjects factors of visit, trial position, and electrode. Data from a group of 16 age-matched normal controls, each of whom will have only underwent one ERP recording, will be used to demonstrate the normal habituation effect. Linear regression will be used to examine the correlations between changes (1-year follow-up minus baseline) in the |count-hit| discrepancy and in ERP measures for which significant treatment effects are shown. Correlations between local peak amplitudes of P2 (measured after application of a 30 Hz low-pass filter) and CGG repeats will be tested.

### Example 9

### Prospective Assessment of the Efficacy of Flupirtine in Patients with Rett Syndrome

This study is designed to determine whether flupirtine or a pharmaceutically acceptable salt thereof will lead to an improvement in one or more symptoms of Rett syndrome. Participants are randomized into 6 separate treatment groups (A-F). Inclusion criteria for randomization will require that each participant has been previously diagnosed with Rett syndrome by clinical evaluation or that the participant is diagnosed with one or more of the essential and supportive criteria for Rett syndrome. Genetic testing may also be used to assist in confirming diagnosis of Rett syndrome. Of all cases of clinically diagnosed Rett syndrome, between 80-97% are found to have mutations in the MECP2 gene (a “positive” genetic test).

Essential Criteria include:
a period of normal development until between 6 to 18 months
repetitive hand movements including hand washing, hand wringing and hand clasping
a normal head circumference at birth followed by a slowing of the rate of head growth with age (starting between the time a child is 6 months and 4 years old)
significantly impaired expressive and receptive language
shakiness of the torso, which also may involve the limbs, particularly when the child is upset or agitated
unsteady, wide-based, stiff-legged gait and sometimes toe walking
Supportive Criteria include:
seizures
breathing irregularities such as apnea, hyperventilation and air swallowing
abnormal sleep patterns and irritability
muscle rigidity or spasticity
irritability or agitation
electroencephalogram (EEG) abnormalities
scoliosis (curvature of the spine)
chewing and/or swallowing difficulties
teeth-grinding
decreased body fat and muscle mass
poor circulation of the lower extremities with cold and bluish-red feet and legs
decreased mobility with age

After randomization the participants are placed into 6 separate treatment groups (A-F) and a placebo group. Treatment group A receives 100 mg flupirtine orally in the morning for two weeks. Treatment group B receives 100 mg flupirtine intravenously in the morning for two weeks. Treatment group C receives 400 mg flupirtine in the evening for two weeks. Treatment group D receives 100 mg flupirtine orally in the morning and 100 mg flupirtine orally in the evening for two weeks. Treatment group E receives 100 mg flupirtine intravenously in the morning and 100 mg flupirtine intravenously in the morning for two weeks. Treatment group F receives 400 mg flupirtine orally in the morning and 400 mg flupirtine in the evening for two weeks.

Participants are assessed throughout the treatment period to determine whether flupirtine administration leads to an improvement in one or more symptoms of Rett syndrome. Several behavioral domains; communication, attention, maladaptive behaviors, and hyper-excitability are assessed. To quantify the communication behavior, participants engage in an unstructured play session to elicit speech and non-verbal communication attempts. Speech attempts by the child are transcribed phonetically and categorized into five different types of vocalizations using the Stark Assessment of Early Vocal Development-Revised (SAEVD-R) (Nathani, Ertmer et al. 2006) which categorizes non-speech and pre-speech sounds (protophones), as well as vowels, consonants and syllables.

Gait abnormalities occur in many cases of Rett syndrome. Thus, five primary spatiotemporal parameters are analyzed: cadence, gait velocity, stride width, step length and percent stance. For each parameter, a principal component analysis is used to establish a gait index for assessment of the subjects.

In addition, primary outcome measures that may be assessed include changes in raw or standard scores between baseline and after trial completion of:
VI. Bayley Scales of Infant and Toddler Development, 3rd edition (or the Mullen Scales of Early Learning in the more developmentally advanced subjects);
VII. Vineland Adaptive Behavior Scales, 2nd edition (standard scores only);
VIII. Preschool Language Scale, 4th edition;
IX. Aberrant Behavior Checklist—Community version; and
X. A change from baseline in the Clinical Global Impressions Severity Scale Score.

Secondary outcome measures may include normalization of the electroencephalogram (EEG) signature when comparing post flupirtine administration results to baseline results.

Those skilled in the art will recognize, or be able to ascertain using no more than routine experimentation, many equivalents to the embodiments and examples described herein. Such equivalents are intended to be encompassed by the claims.

What is claimed is:

1. A method of treating autism comprising administering to a patient in need thereof a pharmaceutical composition comprising gaboxadol or a pharmaceutically acceptable salt thereof.

2. The method of claim 1, wherein the patient is administered about 0.05 mg to about 30 mg gaboxadol or a pharmaceutically acceptable salt thereof.

3. The method of claim 1, wherein the method provides improvement in one or more symptoms of autism in the patient.

4. The method of claim 3, wherein the improvement is provided for more than 6 hours after administration.

5. The method of claim 3, wherein the improvement is provided for at least 8 hours after administration.

6. The method of claim 3, wherein the improvement is provided for at least 12 hours after administration.

7. The method of claim 1, wherein the composition comprises about 1 mg to about 20 mg gaboxadol or a pharmaceutically acceptable salt thereof.
